(19) United States
(12) Patent Application Publication
(10) Pub. No.: US 2020/0138772 A1
(43) Pub. Date: May 7, 2020

(54) STABILIZED FORMULATIONS OF CANNABINOID COMPOSITIONS

(71) Applicant: Volker Berl, New York, NY (US)

(72) Inventors: Volker Berl, New York, NY (US); Chunxin Xia, Flemington, NJ (US)

(21) Appl. No.: 16/506,279

(22) Filed: Jul. 9, 2019

Related U.S. Application Data
(60) Provisional application No. 62/695,276, filed on Jul. 9, 2018.

(51) Int. Cl. A61K 31/352; A61K 31/05; A61K 47/44; A61K 9/00

(52) U.S. Cl. CPC A61K 31/352; A61K 9/0095; A61K 47/44; A61K 31/05

(57) ABSTRACT

In one embodiment, the present application discloses powder and aqueous formulations comprising a stabilized, aqueous purified cannabis oil emulsion comprising: a) CBD and THC wherein the ratio of CBD:THC by wt/wt is from 1,050:1 to 1:1,050, and b) at least one emulsifier selected from the group consisting of Poloxamer 188, Polysorbate 80, Polysorbate 20, Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; and the uses in the treatment of diseases.

### RELATED APPLICATIONS

The present application claims priority to U.S. Provisional Patent Application No. 62/695,276 filed on Jul. 9, 2018, entitled Stabilized Formulations of Cannabinoid Compositions.

### BACKGROUND OF THE INVENTION

The marijuana plant, also known as hemp or cannabis, has been used throughout agricultural history as a source of an intoxicant, medicine and fiber. The medical use of marijuana or cannabis is deeply rooted in history. For almost 5,000 years, cannabis and its medicinal preparations from Cannabis indica and C. sativa have been used for treating nausea, inflammation, vomiting and pain.

The most talked about health risk associated with cannabis is its potential to promote abuse and addiction, especially when it comes to smoking. Therefore, controversies regarding legal, medicinal and ethical use of cannabis have increasingly placed this plant in the spotlight. In 1999, the Office of National Drug Control Policy funded a study by the Institute of Medicine to evaluate medicinal cannabis. The most important component of the cannabis plant is a group of plant chemicals called cannabinoids or phytocannabinoids, which give the cannabis plant medical and recreational properties. The Institute of Medicine recommended testing alternative cannabinoid delivery systems to smoking, and conducting clinical trials to assess efficacy of synthetic and plant-derived cannabinoids for treatment of spasticity, movement disorders, glaucoma and other indications.

Cannabinoids are a class of diverse chemical compounds that act on cannabinoid receptors in cells that alter neurotransmitter release in the brain. So far, over a hundred cannabinoids have been identified from cannabis, of which the two most prominent and intensively studied are tetrahydrocannabinol (THC) and Cannabidiol (CBD).

CBD, a cannabinoid constituent of cannabis plants possesses anxiolytic, antipsychotic, antiemetic and anti-inflammatory properties, without exhibiting the psychoactive effects of Δ⁹-tetrahydrocannabinol (Δ⁹-THC). Δ⁹-THC and CBD are biosynthesized as Δ⁹-tetrahydrocannabinolic acid and cannabidiolic acid from the common precursor olivetol. Both Δ⁹-THC and CBD exert their effects by interacting with the G protein-coupled cannabinoid receptors (GPCRs), CB1 and CB2 with varying affinities. While CB1 receptors are expressed in large quantities in the brain and regions in the central nervous system, and in lower amounts in peripheral tissues; the less studied CB2 receptors have been identified to be localized to immune cells, tonsils and the spleen. The CB1 receptors have been identified to play significant roles in pain perception, memory, motor regulation, appetite, mood and sleep, whereas the CB2 receptors have been linked with anti-inflammation, pain reduction and reducing tissue damage. Physiologically, upon activation by the endocannabinoids like anandamide and 2-arachidonylglycerol (2-AG) (which are short lived), CB1 and CB2 trigger a downstream cascade of events that mediate homeostasis and healthy functioning. In contrast, the phytocannabinoids Δ⁹-THC and CBD that directly or indirectly interact with CB1 and CB2 with varying affinities modulate the activities of these receptors for prolonged durations.

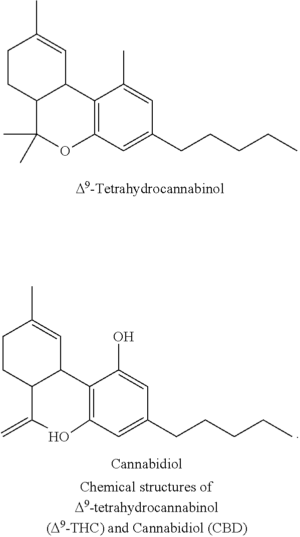 [SMILES: CCCCCc1cc(C)c2c(c1)OC(C)(C)C1CCC(C)=CC21 ; C=C1Oc2cc(CCCCC)cc(O)c2C2C=C(C)CCC12]

Δ⁹-THC is the major psychoactive cannabinoid and mimics the action of the endogenous cannabinoid receptor ligands anandamide and 2-AG by activating both CB1 and CB2 receptors. Due to its binding to CB1 receptors which are specifically present in the central nervous system in areas associated with pain (eg. spinal trigeminal nucleus, amygdala, basal ganglia and periaqueductal gray), Δ⁹-THC possesses antinociceptive activity and is hence used as an analgesic agent in certain pain medications. In addition, Δ⁹-THC has also been shown to be effective in the treatment of glaucoma, nausea, chronic pain, multiple sclerosis, epilepsy and inflammation in several pre-clinical and clinical studies. However, Δ⁹-THC abuse is a global concern and due to the behavioural and psychological dependence, Δ⁹-THC has remained a subject of controversy.

CBD, which is the non-psychoactive phytocannabinoid, and can hence be a promising therapeutic, has gained increasing attention in the recent past. Previous studies have shown that CBD is a promising potential therapeutic for various disorders of the central nervous system including anxiety, epilepsy, schizophrenia, Parkinson's disease, Alzheimer's disease, multiple sclerosis and many more. Unlike Δ⁹-THC, CBD does not activate CB1 and CB2, and instead blocks the cannabinoids that activate these receptors by a complex mechanism. Several groups have proposed that this activity not only results in the non-psychotropic effects exhibited by CBD but may also account for ameliorating some of the psychotropic effects shown by Δ⁹-THC. In addition, by lowering the psychoactivity of Δ⁹-THC, CBD may also potentiate some of Δ⁹-THC's benefits by enhancing its tolerability and widening its therapeutic window. CBD can also inhibit or delay the reuptake and hydrolysis of the endocannabinoids like anandamide and adenosine. CBD has also been hypothesized to interact with several other non-endocannabinoid signaling systems such as serotonin receptors, vanilloid receptors, GPR-55 (orphan receptors), peroxisome proliferator activated receptors (PPARs) making it a “multi-target drug”. In addition to these activities, the polyphenolic ring in CBD also results in it being a potent antioxidant. All these results have prompted the exploration of the therapeutic potential of CBD for a range of neuropsychiatric as well as inflammatory disorders.

Given the breadth of therapeutic benefits, it would be advantageous to better understand the pharmacokinetics of cannabinoids, and to develop suitable optimized delivery systems in which cannabinoids are transported systemically to achieve a therapeutically effective dose. Cannabis products are commonly either inhaled by smoking a cannabis cigarette, taken orally as capsules or in baked foods or liquids. Various other routes of administration and delivery forms have been tested for therapeutic purposes. The rectal route with suppositories has been applied in some patients, and dermal and sublingual, and topical administration are also under scientific investigations.

As the oral route is the most commonly acceptable delivery passway for drug and nutraceauticals absorption, several researchers have attempted to study the pharmacokinetics and pharmacodynamics of CBD and Δ⁹-THC. However, as researches have previously shown, systemic bioavailability of oral CBD in humans was only 6%, much lower than smoked CBD, which was around 31%; and bioavailability of oral and smoked Δ⁹-THC is shown to be 4-12% and 10-27% respectively. Other studies have previously determined the time to achieve peak plasma concentration (tₘₐₓ) as 1.5-4 h for different doses of CBD and 1-2 h for different doses of Δ⁹-THC. In several studies, maximal plasma concentrations of orally taken Δ⁹-THC were observed as late as 4 hours and even 6 hours in some cases. The half-life of CBD in humans was found to be between 18-33 h upon intravenous injection, 27-35 h upon smoking, and 2-5 days upon oral administration.

Lipid solubility and molecular size are the major limiting factors for molecules to pass the biological membrane and to be absorbed systematically following oral or topical administration. High lipophilicity of cannabinoids results in poor dissolution in the aqueous environment of the gastriointestinal tract, and thus makes this class of compounds poorly absorbed systemically from oral dosage forms. Therefore, the low bioavailability of oral cannabinoids restricts their therapeutic and supplement uses.

Currently, there are only a few approved oral formulations of cannabinoids becoming commercially available for treating nausea, vomiting associated with cancer, multiple sclerosis, intractable cancer pain, etc. They are: Dronabinoil, available commercially as Marinol®™ soft gelatin capsules and Namisol®™ as sublingual tablets have been approved by Food and Drug Administration (FDA). Nabilone as Cesamet® has been marketed in Canada, the United States, the United Kindom and Mexico. Sativex®, a mouth spray containing THC and CBD, has been proven to treat spasticity due to multiple sclerosis.

However, a bioavailability study of Sativex® indicated that the mean Cₘₐₓ values recorded were still well below those reported in patients who smoked/inhaled cannabis, which means this commercial product did not improve the bioavailability to a desirable extent. In terms of formulation, these products are either capsules or sprays/tinctures, generally composed of simple solutions of cannabis extracts in non-aqueous or lipohilic carrier liquids, lacking a suitably designed drug delivery system otherwise customary to the pharmaceutical oral delivery of poorly soluble drugs. Moreover, these existing drug formulations can only be used as medical products, and cannot be applied directly and broadly to food, beverages or other edibles, or topicals. Thus, there is a need for novel and effective oral formulation of cannabinoids that can be applied not only to pharmaceutical, but also food, beverage and supplement applications.

Emulsions have been widely used as flavor, drug or nutraceutical delivery systemes in beverage, cosmetics, or pharmaceutical for decades. Typical emulsions are a perfect mixture of water and oil with the help of amphiphilic surfactants, and oil in water (O/W) emulsions can carry lipophilic compounds into suspended small oil droplets and makes them “water-soluble”. Given the lipophilic property of cannabinoids, emulsions are likely to be a desirable delivery systems for them. Nevertheless, the limited thermodynamic stability of emulsions remains a practical application issue, which means that with time they will undergo coalescence, creaming, sedimentation, Ostwald ripening and finally separation into their two original liquid phases. This represents the biggest challenge in existing commercial applications.

Unlike emulsions, microemulsions, including micellar solutions, are usually thermodynamically stable and sometimes transparent/translucid dispersions that form spontaneously without the need of high shear energy input, when the compounds thereof are properly mixed with each other under the right conditions. The dispersed oil droplets in microemulsions can be less than 100 nm in diameter, which makes them transparent to visible light as a result of minimal visible light scattering. As a result, microemulsions can appear as clear or translucid isotropic solutions, when the formulation composition and conditions are designed properly. Since microemulsions offer the advantage of spontaneous formation, ease of manufacturing and scale-up, thermodynamic stability, and improved drug solubilization and potential for improved oral bioavailability, they are considered as the most ideal delivery systems for encapsulating cannabinoids.

A classical oil-in-water microemulsion consists of water, a co-solvent, oil and one or more surfactants as co-surfactants. The selection of the components, the proportion of each ingredient, and the methods of emulsification are critical for their formation, as well as the final characteristics such as optical appearance, and the organoleptic and thermodynamic time-stability of the microemulsion. In addition, when such microemulsions are used as nutraceuticals or drug delivery systems in foods and beverages, they may lead to excellent shelf-life stability and a significant relative bioavailabitity increase via oral administration for the encapsulated cannabinoids. One objective of the present invention is to prepare optimized cannabinoid emulsion compositions that lead to:
emulsion shelf stability in terms of physicochemical emulsion decomposition (i.e., avoiding coalescence, Ostwald ripening, etc.), for an ingredient, and when added to a finished food or beverage product;
compatibility of the emulsion with customary food and beverage finished product bases and formulations;
favorable organoleptic and sensory properties and ease of flavoring finished products to suppress typical cannabinoid extract flavor notes;
ease of emulsion manufacturing at commercial scale without the need of high shear/high energy intake or specialized equipment;
enhanced relative oral bioavailability, supported by a Phase I clinical pharmacokinetic or pharmacodynamics study to evaluate the effectiveness of such a delivery system, with the aim of providing an improved and standardized dose delivery to patients and consumers with a faster onset of effects;
highest possible cannabinoid loading;
sub 100 nm particle size conveying substantial clarity/translucidity to the emulsion,
lowest possible emulsifier and emulsifier mixture concentrations and
protection of the emulsified cannabinoids from oxidative processes assuring end-of-shelf-life bioactive stability.

### SUMMARY OF THE INVENTION

A need exists for novel methods of preparing and delivering stabilized formulations comprising CBD and THC compositions as pharmaceutical or nutraceutical products, food and beverages, optionally containing other nutritional products. In one embodiment, the present application discloses cannabidiol (CBD) and tetrahydrocannabinol (THC, dronabinol or more precisely its main isomer (−)-trans-Δ⁹-tetrahydrocannabinol) as pharmaceutical or nutraceutical products, food and beverages, optionally containing other nutritional products. In one aspect, the formulations are oxidatively stable, are water soluble, and provide increased bioavailability that allows for effective uptake by the body.

In one embodiment, there is provided a stable emulsion comprising a CBD oil or THC oil (or CBD and/or THC, or referred to as CBD/THC), or CBD/THC mixtures. In another embodiment, there is provided a stable, substantially clear, water soluble formulation of CBD, THC or CBD/THC mixtures.

In one embodiment, the present application discloses a stabilized, aqueous cannabis oil emulsion comprising: a) CBD and THC wherein the ratio of CBD:THC by wt/wt is from 1,050:1 to 1:1,050, and b) at least one emulsifier selected from the group consisting of Poloxamer 188, Polysorbate 80, Polysorbate 20, Vit E-TPGS (TPGS), TPGS-1000, TPGS-750-M, Solutol HS 15, PEG-40 Hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; wherein the emulsion is stable for a period of at least 30 days when stored at about 20-30° C. In one aspect, the CBD, THC or mixtures of CBD and THC are isolates. In another aspect, the cannabis oil is purified. As used herein, the purification of the cannabis oil may be performed using distillation such as distillation under reduced pressure, chromatography such as HPLC, crystallization, and a combination thereof. In one variation, the purified CBD and THC in the cannabis oil is greater than 25% in the cannabis oil, greater than 30%, greater that 35% or greater than 40% in the cannabis oil.

In one aspect, the emulsion is stable toward oxidation for a period of at least 30 days when stored at about 20-30° C. In one aspect of the emulsion, the ratio of CBD:THC by wt/wt ranges from about 1,050:1 to 350:1, 350:1 to 300:1, 300:1 to 250:1, 250:1 to 200:1, 150:1 to 100:1, 100:1 to 50:1, 50:1 to 20:1, 20:1 to 1:20, 15:1 to 1:15, 1:1 to 1:10, 11:1 to 1:10, 5:1 to 1:5 or about 3:1 to 1:3. In another aspect of the emulsion, the ratio of CBD:THC by wt/wt is about 1,050:1, 750:1, 500:1, 350:1, 300:1, 250:1, 200:1, 150:1, 100:1, 50:1, 40:1, 30:1, 20:1, 15:1, 10:1, 5:1, 4:1, 3:1, 2:1 or about 1:1. In another aspect of the emulsion, the ratio of CBD:THC by wt/wt is about 1:1,050, 1:750, 1:500, 1:350, 1:300, 1:250, 1:200, 1:150, 1:100, 1:50, 1:30, 1:20, 1:15, 1:10, 1:5:, 1:4, 1:3, or about 1:2. In another aspect, the emulsion has an overall CBD and THC concentration of about 10% to 15%, 15% to 25%, 25% to 35%, 35% to 45%, 45% to 55%, 55% to 65%, 65% to 70%, 70% to 75%, 75% to 80%, 80% to 85%, 85% to 90%, 90% to 95%, 95% to 97%, 97% to 98% or about 99% or more.

In another aspect of the above emulsion, the emulsifier is GRAS or a food grade emulsifier. In another aspect, the ratio of CBD:THC is 1:1. In one aspect, the purity of CBD is from about 20% to 98%, such as 30%, 40%, 50%, 60%, 70%, 80%, 90% or about 98%. In another aspect, the purity of the CBD is about 99%, 98%, 97%, 95%, 93%, 92%, 90%, 85%, 80% 70%, 60%, 50%, 40%, 30%, 20%, 10% or about 5%. In another aspect, the purity of THC oil is from about 20% to 95%, such as 30%, 40%, 50%, 60%, 70%, 80% or about 90%. In another aspect, the purity of the THC oil is about 99%, 98%, 97%, 95%, 93%, 92%, 90%, 85%, 80% 70%, 60%, 50%, 40%, 30%, 20%, 10% or about 5%. Natural CBD and THC oils are typically refined, purified, distilled or extracted from plant sources, and may also contain other natural oils. The purity of the CBD and THC noted above may be determined by HPLC methods or by gas chromatography.

In another aspect, the emulsion, such as a water emulsion, further comprises one or more co-solvents selected from the group consisting of ethanol, glycerol, propylene glycol, 1,3-propanediol, butylene glycol, erythritol, xylitol, mannitol, sorbitol, isomalt, polyethylene glycols (PEG)-400, and a combination thereof. In another aspect, the emulsion further comprises one or more vegetable oils selected from the group consisting of arachis oil, olive oil, sesame oil or coconut oil and a mineral oil, rice bran oil, or combinations thereof.

In yet another aspect, the emulsion further comprises one or more oil selected from the group consisting of Cannabis oil (hemp oil), cottonseed oil, soybean oil, sunflower oil, castor oil, corn oil, olive oil, palm oil, peanut oil, almond oil, sesame oil, rapeseed oil, peppermint oil, canola oil, palm kernel oil, hydrogenated soybean oil, medium-triglyceride, short-chain triglyceride, glyceryl esters of saturated fatty acids, glyceryl behenate, glyceryl distearate, glyceryl isostearate, glyceryl laurate, glyceryl monooleate, glyceryl monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and combinations thereof.

In another aspect, the emulsion further comprises one or more masking or flavoring component selected from the group consisting of natural cinnamon oil, peppermint oil, clove oil, bay oil, thyme oil, artificial, natural or synthetic fruit flavors selected from the group consisting of vanilla, chocolate, coffee, cocoa, and citrus oil selected from the group consisting of lemon, lime, orange, grape, grapefruit, and fruit essences selected from the group consisting of apple, pear, peach, strawberry, watermelon, raspberry, cherry, plum, pineapple and apricot, or combinations thereof.

In another aspect, the emulsion further comprises one or more additives comprising: a) a stabilizer or antioxidant selected from the group consisting of tocopherols, flavonoids, catechins, superoxide dismutase, lecithin, gamma oryzanol, vitamins A, C (ascorbic acid) and E including homologues and isomers thereof, camosol, carnosic acid and rosmanol, hawthorn extract and proanthocyanidins, or combinations thereof; and b) a reducing agent selected from the group consisting of L-ascorbic acid-6-palmitate, vitamin C and ubiquinol, or mixtures thereof. In another aspect, the emulsion further comprises a metal chelator selected from the group consisting of ethylenediaminetetraacetic acid (EDTA), disodium EDTA and calcium disodium EDTA and mixtures thereof.

In another aspect of the emulsion, the range of the ratio of the emulsifier to CBD/THC oil is between 11.0:1.0 to 1.0:1.0, 7.0:1.0 to 1.5:1.0 or 5.0:1.0 to 2.0:1.0. In one aspect, the ratio of the emulsifier to CBD/THC oil is between about 15:1 to about 10:1, 10:1 to 5:1, 5:1 to 3:1, 3:1 to 2:1, or about 2:1 to 1:1. In one aspect of the emulsion, the ratio of the emulsifier to CBD/THC oil is between about 15:1, 13:1, 12:1, 10:1, 9:1, 8:1, 7:1, 6:1, 5:1, 4:1, 3:1, 2:1 or about 1:1. In another aspect of the emulsion, the CBD/THC oil concentration in the emulsion is about 10%, 9%, 8%, 7%, 5%, 3%, 2%, 1%, 0.5%, 0.1% or 0.01% or less. In one aspect, the range of the CBD/THC oil concentration in the emulsion is from about 0.01% to 10% w/w, 1%-9% or about 2%-6%.

In another aspect, the emulsion comprises of particle size that is less than about 500 nm, less than 300 nm, less than 200 nm, less than 100 nm, less than 80 nm, less than 60 nm; less than 40 nm; or between about 20 and 30 nm, as measured by DLS or cryo-TEM. In another variation, the emulsion comprises of particle size that is in the range of about 20 nm to 80 nm, 20 nm to 40 nm, 40 nm to 60 nm and about 60 nm to 80 nm. In another aspect, the emulsion has a measured Nephelometric Turbidities in a range of about 10 to 1000, 20 to 300 or 30 to 100.

In another embodiment, there is provided a method for the preparation of any one of the above emulsions, the method comprising: a) weighing the components of the above emulsion into a reaction container; b) heating the combined emulsion to a temperature from about 25° C. to about 130° C. with agitation for a sufficient amount of time to prepare the emulsion; and c) cooling the emulsion to about 25° C. In another aspect, the preparation is performed under a nitrogen or other inert atmosphere. In yet another aspect, the heating of the emulsion is performed to a temperature of about 70° C. to 100° C., or about 80° C. to 95° C. In another aspect, the cooling of the emulsion is performed using an external ice bath or equivalent. In another aspect of the above emulsion prepared by the cited method, the resulting stable emulsion has a shelf stability of at least 3 months, 6 months or 12 months when stored at about 0° C. to 50° C., or about 25° C. to 35° C. In one aspect of the emulsion, resulting stable emulsion has a shelf stability of at least 3 months, 6 months or 12 months when stored at about 0-50° C., 10-40° C. or 20-30° C. In another aspect, of the above emulsion, the natural odor of the CBD/THC emulsion is effectively masked and provides a pleasant taste for oral consumption. In another aspect of the emulsion, the oxidative stability of the emulsion is enhanced over that of a CBD/THC mixture by at least 3 months when the CBD/THC mixture is stored at about 0° C. to 50° C.

In another embodiment, there is provided a liquid nutritional composition selected from the group consisting of beverages, soft drinks, carbonated beverages, enhanced waters, gels, gelatins, concentrates, beverage enhancers, wherein the composition is prepared by the method comprising: a) obtaining an emulsion of the above; and b) diluting the emulsion to a desired liquid nutritional composition.

In another embodiment, the water-soluble formulation further comprises a water soluble antioxidant. In another embodiment, the water-soluble formulation further comprises a metal chelator. In another embodiment, the water-soluble formulation further comprises a water-soluble reducing agent. In another embodiment, the water-soluble formulation further comprises a lipophilic antioxidant. In yet another embodiment, the water-soluble formulation further comprises a lipophilic reducing agent, or a combination of each of the above.

In one variation, the solubilizing agent is selected from the group consisting of solubilizing agents having a hydrophilic-lipophilic balance (HLB) of 8-18, HLB of 7-9 and HLB of 8-12, HLB of 13-15, TPGS (polyoxyethanyl-a-tocopheryl succinate) and combinations thereof. In another aspect, the solubilizing agent is TPGS (polyoxyethanyl-a-tocopheryl succinate), TPGS-750-M or TPGS-1000 (D-alpha-tocopheryl polyethylene glycol 1000 succinate), wherein the tocopheryl is the natural tocopherol isomer, the unnatural tocopherol isomer, or the corresponding racemic material. In another aspect, the water soluble reducing agent is L-ascorbic acid-6-palmitate. In another embodiment, the metal chelator is ethylenediamine tetraacetic acid (EDTA). In another embodiment, the reducing agent is sodium bisulfite.

In another embodiment, there is provided a method for stabilizing a substantially water insoluble CBD/THC mixture in an aqueous solution comprising contacting the CBD/THC mixture with a composition comprising a micelle-forming surfactant, a water-soluble reducing agent, and a metal chelator in water, at an elevated temperature, and for a sufficient period of time to dissolve the CBD/THC mixture. In another aspect, the micelle-forming surfactant is TPGS, TPGS-750-M or TPGS-1000. In one variation, the metal chelator ethylenediamine tetraacetic acid. In another variation, the method further comprises contacting the aqueous solution with a metal bisulfite reducing agent.

In one embodiment, there is provided a stabilized aqueous formulation comprising a substantially water insoluble CBD/THC mixture, a micelle-forming surfactant, a water soluble reducing agent, a metal chelator and a reducing agent, wherein the formulation remains substantially clear and stable when stored at or below room temperature for a period of at least 6 months or at least 12 months. In another embodiment, there is provided a stabilized food, beverage, pharmaceutical or nutraceutical product comprising the aqueous formulation of the above.

### BRIEF DESCRIPTION OF THE FIGURE

FIG. 1 is a representative graph indicative of the particle size distribution associated with a formulation of the present application.

### DETAILED DESCRIPTION OF THE PRESENT APPLICATION

### Definitions

Unless specifically noted otherwise herein, the definitions of the terms used are standard definitions used in the art of organic synthesis and pharmaceutical sciences. Exemplary embodiments, aspects and variations are illustrative in the figures and drawings, and it is intended that the embodiments, aspects and variations, and the figures and drawings disclosed herein are to be considered illustrative and not limiting.

The term “absorption enhancer” usually refers to an agent whose function is to increase absorption by enhancing membrane permeation, rather than increasing solubility, so such agents are sometimes more specifically termed permeation enhancers.

The term “cannabinoid” is defined as one of a class of diverse compounds that acts on cannabinoid receptors in cells that alter neurotransmitter release in the brain. Cannabinoid may comprise all ligands of the cannabinoid receptor and related compounds, including the endocannabinoids (produced naturally in the body by animals), the phytocannabinoids (found in cannabis and some other plants), and synthetic cannabinoids that may be manufactured artificially.

In one aspect, the term “cannabidiol” or “CBD” is a phytocannabinoid that is one of at least 110 active cannabinoids that have been identified in cannabis. CBD may account for up to 40% of the plant's extract, and have been considered to provide a large scope of potential medicinal applications. In another aspect, the term “cannabinoid” is a compound (such as cannabinol, THC or cannabidiol) that is found in the plant species Cannabis saliva (marijuana), and includes metabolites and synthetic analogues thereof, that may have psychoactive properties. Cannabinoids include compounds, such as THC, that have high affinity for the cannabinoid receptor, and compounds that do not have significant affinity for the cannabinoid receptor, such as cannabidiol (CBD). Cannabinoids also include compounds that have a characteristic dibenzopyran ring structure (such as in THC) and cannabinoids which do not have a pyran ring (such as cannabidiol). Cannabinoids also includes, for example, THC, CBD, dimethyl heptylpentyl cannabidiol (DMHP-CBD) and 6,12-dihydro-6-hydroxy-cannabidiol (see U.S. Pat. Nos. 5,227,537 and 4,876,276); and cannabidiol (−)(CBD) analogs. The most notable cannabinoid is the phytocannabinoid tetrahydrocannabinol (THC), the primary psychoactive compound in cannabis. Cannabidiol (CBD) is another major constituent of the plant. There are at least 113 different cannabinoids isolated from cannabis, exhibiting varied effects.

The phytocannabinoids have been numbered according to the monoterpenoid system or the dibenzopyran system. A total of phytocannabinoids have been identified, most of them belonging to several subclasses or types: the cannabigerol (CBG), cannabichromene (CBC), cannabidiol (CBD), Δ⁹-THC, Δ⁸-THC, cannabicyclol (CBL), cannabielsoin (CBE), cannabinol (CBN), cannabinodiol (CBDL) and cannabitriol (CBTL) types. A total of nine cannabinoids belong to the Δ⁹-THC group, with side chains of one, three, four and five carbons. As provided herein, the compositions and formulations of the present application comprising CDB, THC or CBD/THC, as disclosed herein, may also be replaced by the cannabinoids or mixtures of cannabinoids, as recited in the following table, to provide active formulations.

| Cannabinoid Structure | Representative Cannabinoid | |
|---|---|---|
| Types | Name | Chemical Structure |
| Cannabidiol-Type | (−)-Cannabidiol (CBD) | 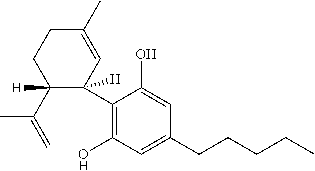 [SMILES: C=C(C)[C@@H]1CCC(C)=C[C@H]1c1c(O)cc(CCCCC)cc1O] |
| | Cannabidiolic acid (CBDA) | 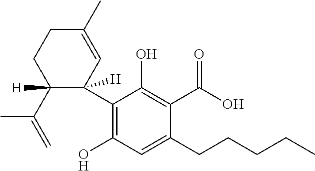 [SMILES: C=C(C)[C@@H]1CCC(C)=C[C@H]1c1c(O)cc(CCCCC)c(C(=O)O)c1O] |
| | Cannabidiol monomethylether (CBDM) | 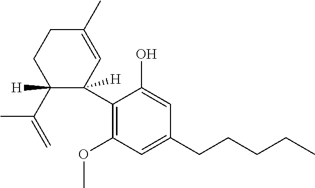 [SMILES: C=C(C)[C@@H]1CCC(C)=C[C@H]1c1c(O)cc(CCCCC)cc1OC] |
| Tetrahydrocannabinol- Type | Δ⁹-Tetrahydrocannabinol (Δ⁹-THC) | 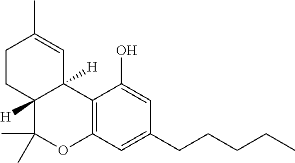 [SMILES: CCCCCc1cc(O)c2c(c1)OC(C)(C)[C@@H]1CCC(C)=C[C@@H]21] |
| | Δ⁹-Tetrahydrocannabinolic acid A (Δ⁹-THCA A) | 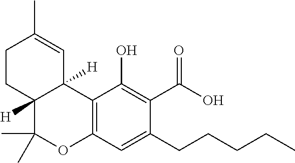 [SMILES: CCCCCc1cc2c(c(O)c1C(=O)O)[C@@H]1C=C(C)CC[C@H]1C(C)(C)O2] |
| | Δ⁹-Tetrahydrocannabinolic acid B (Δ⁹-THCA B) | 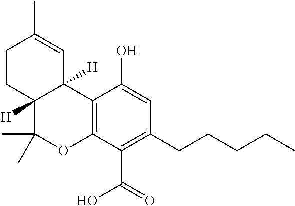 [SMILES: CCCCCc1cc(O)c2c(c1C(=O)O)OC(C)(C)[C@@H]1CCC(C)=C[C@@H]21] |
| | (−)-Δ⁸-trans-(6aR,10aR)- Δ⁸-Tetrahydrocannabinol (Δ⁸-THC) | 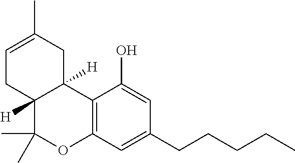 [SMILES: CCCCCc1cc(O)c2c(c1)OC(C)(C)[C@@H]1CC=C(C)C[C@@H]21] |
| | (−)-Δ⁸-trans-(6aR,10aR)- Tetrahydrocannabinolic acid A (Δ⁸-THCA A) | 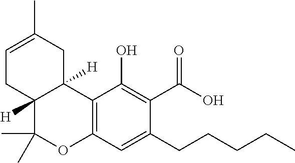 [SMILES: CCCCCc1cc2c(c(O)c1C(=O)O)[C@@H]1CC(C)=CC[C@H]1C(C)(C)O2] |
| Cannabinol-Type | Cannabinol (CBN) | 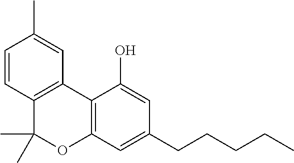 [SMILES: CCCCCc1cc(O)c2c(c1)OC(C)(C)c1ccc(C)cc1-2] |
| | Cannabinol acid A (CBNA) | 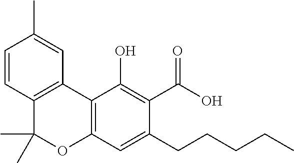 [SMILES: CCCCCc1cc2c(c(O)c1C(=O)O)-c1cc(C)ccc1C(C)(C)O2] |
| | Cannabinolmethylether (CBNM) | 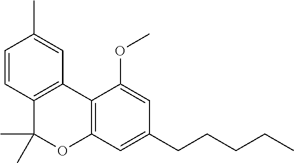 [SMILES: CCCCCc1cc(OC)c2c(c1)OC(C)(C)c1ccc(C)cc1-2] |
| Cannabigerol-Type | Cannabigerol ((E)-CBG) | 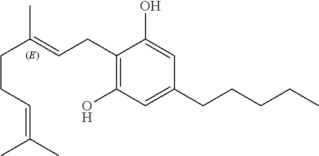 [SMILES: CCCCCc1cc(O)c(C/C=C(\C)CCC=C(C)C)c(O)c1] |
| | Cannabigerolic acid A ((E)-CBGA A) | 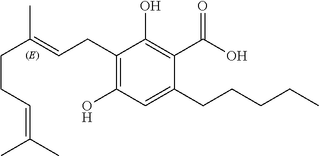 [SMILES: CCCCCc1cc(O)c(C/C=C(\C)CCC=C(C)C)c(O)c1C(=O)O] |
| Cannabichromene-Type | (±)-Cannabichromene (CBC) | 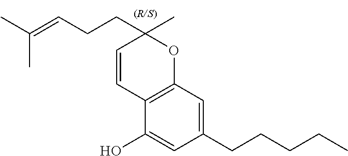 [SMILES: CCCCCc1cc(O)c2c(c1)OC(C)(CCC=C(C)C)C=C2] |
| | (±)-Cannabichromenic acid A (CBCA A) | 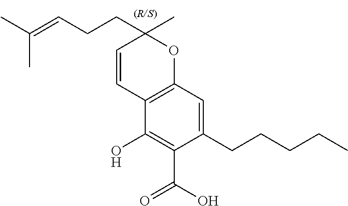 [SMILES: CCCCCc1cc2c(c(O)c1C(=O)O)C=CC(C)(CCC=C(C)C)O2] |
| Canabinodiol-Type | Cannabinodiol (CBND) | 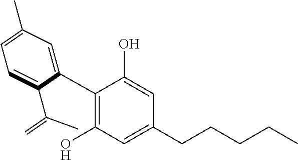 |
| Canabitriol-Type | (−)-(9R,10R)-trans- Cannabitriol ((−)-trans-CBT) | 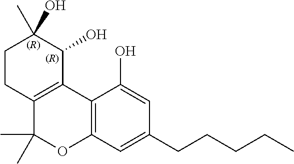 |
| | (+)-(9S,10S)-Cannabitriol ((+)-trans-CBT) | 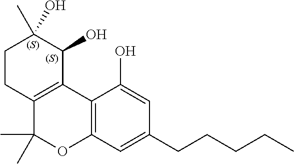 |
| | 8,9-Dihydroxy-Δ^{6a(10a)}- tetrahydrocannabinol (8,9-Di-OH-CBT) | 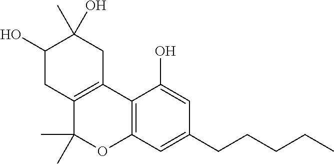 [SMILES: CCCCCc1cc(O)c2c(c1)OC(C)(C)C1=C2CC(C)(O)C(O)C1] |
| Canabielsoin-Type | (5aS,6S,9R,9aR)- Cannabielsoin (CBE) | 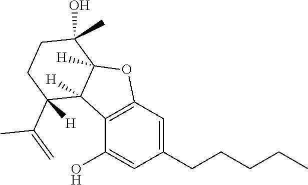 |
| | (5aS,6S,9R,9aR)- Cannabielsoinic acid A (CBEA A) | 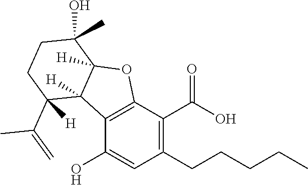 |
| | Cannabielsoinic acid B (CBEA B) | 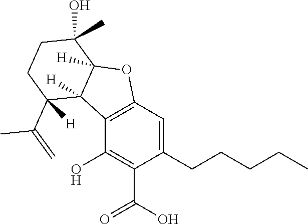 |
| | Dehydrocannabifuran (DCBF) | 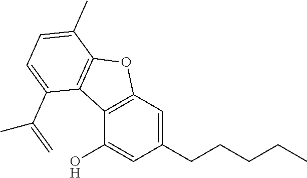 [SMILES: C=C(C)c1ccc(C)c2oc3cc(CCCCC)cc(O)c3c12] |
| | Cannabifuran (CBF) | 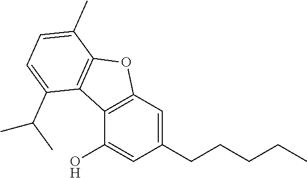 [SMILES: CCCCCc1cc(O)c2c(c1)oc1c(C)ccc(C(C)C)c12] |
| Isocannabinoid-Type | (−)-Δ⁷-trans-(1R,3R,6R)- Isotetrahydrocannabinol | 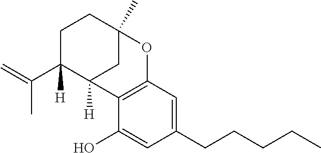 [SMILES: C=C(C)[C@@H]1CC[C@]2(C)C[C@H]1c1c(O)cc(CCCCC)cc1O2] |
| Cannabicyclol-Type | (±)-(1aS,3aR,8bR,8cR)- Cannabicyclol (CBL) | 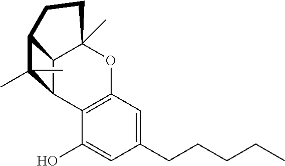 |
| | (±)-(1aS,3aR,8bR,8cR)- Cannabicyclolic acid A (CBLA A) | 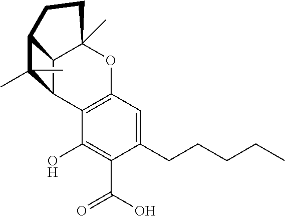 |
| Cannabicitran-Type | Cannabicitrane (CBT) | 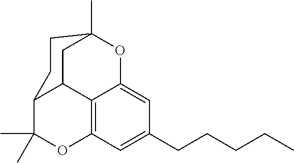 [SMILES: CCCCCc1cc2c3c(c1)OC(C)(C)C1CCC(C)(CC31)O2] |
| Cannabichromanon-Type | Cannabichromanone (CBCN) | 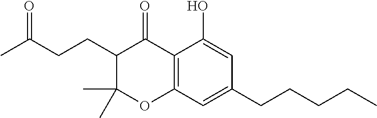 [SMILES: CCCCCc1cc(O)c2c(c1)OC(C)(C)C(CCC(C)=O)C2=O] |
| | Cannabicoumaronone (CBCON) | 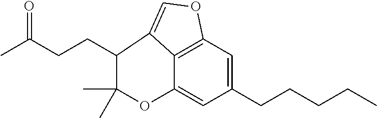 [SMILES: CCCCCc1cc2c3c(coc3c1)C(CCC(C)=O)C(C)(C)O2] |

### Definition and Concentration of CBD in Natural Cannabis:

CBD can occur in up to 40% of the cannabinoid extracts from cannabis. CBD generally occurs in the cannabis plant prior to processing as CBDA which has a carboxylic acid group. The 2-carboxylic acids of the cannabinoids can be decarboxylated by heat, light, or alkaline conditions to their respective decarboxylated compounds.

### Function of CBD and CBDA:

CBD and CBDA have been shown effective in treating inflammation, diabetes, cancer, mood disorders (PTSD to ADD) and neurodegenerative disease such as Alzheimer's. It has been shown to have anti-convulsive, anti-anxiety, anti-psychotic, anti-nausea and anti-rheumatoid arthritic and sedative properties, and a clinical trial showed that it eliminates anxiety and other unpleasant psychological side effects. CBD does not display the psychoactive effects of Δ⁹-THC. CBD was found in one study to be more effective than aspirin for pain relief and reducing inflammation. CBD has been shown to be a potent antioxidant as well as having neuroprotective and anti-inflammatory uses.

Definition of THC: The most potent stereoisomer occurs naturally as Δ⁹-THC where the two chiral centers at C-6a and C-10a are in the trans configuration as the (−)-trans-isomer, and this stereoisomer is also known as dronobinol. There are seven double bond isomers in the partially saturated carboxylic ring including Δ^{6a,7}-THC, Δ⁷-THC, Δ⁸-THC, Δ^{9, 11}-THC, Δ¹⁰-THC and Δ^{6a, 10a}-THC, using the dibenzopyran numbering.

Tetrahydrocannabinol, such as Δ⁹-THC, helps reduce nausea and vomiting, which is particularly beneficial to patients undergoing chemotherapy for cancer. Patients suffering from AIDS often experience a lack of appetite, of which THC is also helpful in counteracting. THC is also useful for glaucoma relief.

In the present application, CBD oils (or CBD oil) refer to cannabis extracts that have CBD as the main or primary bioactive components. Likewise, THC oils refer to cannabis extracts that have THC as the main or primary bioactive components. While commercial CBD oils can vary from 10% to >98% of CBD, in typical CBD oils used in this invention, CBD may vary from 30% to 70%. Likewise, while commercial THC oils can vary from 10 to >98% THC, a more typical commercial range used in this invention is from 60%-90%.

As used herein, the term “CBD and THC” or “CBD/THC” includes substantially pure CBD, substantially pure THC or a mixture of CBD and THC having the particular purity, CBD/THC ratio and concentrations as disclosed in the present application. For example, a composition comprising “CBD and THC” includes at least one of 1) substantially pure CBD without THC, 2) substantially pure THC without CBD, and 3) a mixture of both CBC and THC having the purity, concentrations and ratios as disclosed herein.

Emulsifiers (also referred to as surfactants), are a group of surface-active agents that promote the formation and stabilization of an emulsion. HLB value, which is an abbreviation of Hydrophile-Lipophile Balance, is an empirical expression for the relationship of the hydrophilic (“water-loving”) and hydrophobic (“water-hating”) groups of a surfactant. Emulsifier HLB values range from 1-45, while the range for nonionic emulsifiers typically is from 1-20. The more lipophilic an emulsifier, the lower its HLB value. Conversely, the more hydrophilic an emulsifier, the higher its HLB value. Lipophilic emulsifiers have greater solubility in oil and lipophilic substances, while hydrophilic emulsifiers dissolve more easily in aqueous media. In general, emulsifiers with HLB values greater than 10 or greater than about 10 are called “hydrophilic emulsifiers,” while emulsifiers having HLB values less than 10 or less than about 10 are referred to as “hydrophobic emulsifiers.” HLB values have been determined and are available for most emulsifiers. HLB values for a given emulsifier or co-emulsifier can vary, depending upon the empirical method used to determine the value. HLB values of emulsifiers and co-emulsifiers provide a rough guide for formulating compositions based on relative hydrophobicity/hydrophilicity. For example, an emulsifier typically is selected from among emulsifiers having HLB values within a particular range of the emulsifier or co-emulsifier that can be used to guide formulations. A co-emulsifier is an emulsifier acting together with another emulsifier to enhance its properties; and may be used to further reduce the surface tension of a liquid. Therefore, the emulsion will become further stabilized.

The term “vitamin C derivative” as used herein means any compound that releases ascorbic acid (vitamin C) in vivo or in vitro, as well as solvates, hydrates and salts thereof. The term also includes vitamin C analogs wherein one or more of the hydroxyl groups of vitamin C are substituted with another moiety and wherein the vitamin C analog essentially retains the stabilizing activity of vitamin C in vitro or in vivo.

As used herein, the term “solubilizing agent” is used interchangeably with the term “surfactant” or “emulsifier”. In one embodiment, the solubilizing agent is a nonionic, amphiphilic molecule, wherein the term amphiphilic means that the molecule includes at least one hydrophobic (e.g., lipid-soluble) moiety, such as a moiety derived from a tocopherol, a sterol, or a quinone (or derived hydroquinone, such as in the case of ubiquinone and ubiquinol) and at least one hydrophilic (e.g., water-soluble) moiety, such as polyethylene glycol or a simple sugar, carbohydrate or a carbohydrate derivative.

As used herein, the terms “stabilizer”, and “antioxidant”, are recognized in the art and refer to synthetic or natural substances that prevent or delay the oxidative or free radical or photo-induced deterioration of a compound, and combinations thereof. Exemplary stabilizers include tocopherols, flavonoids, catechins, superoxide dismutase, lecithin, gamma oryzanol; vitamins, such as vitamins A, C (ascorbic acid) and E (tocopherol and tocopherol homologues and isomers, especially alpha and gamma- and delta-tocopherol) and beta-carotene (or related carrotenoids); natural components such as camosol, carnosic acid and rosmanol found in rosemary and hawthorn extract, proanthocyanidins such as those found in grape seed or pine bark extract, and green tea extract. In one variation, the vitamin E includes all 8-isomers (all-rac-alpha-tocopherol), and also include d,l-tocopherol or d,l-tocopherol acetate. In one variation, the vitamin E is the d,d,d-alpha form of vitamin E (also known as natural 2R,4R′,8R′-alpha-tocopherol). In another variation, the vitamin E includes natural, synthetic and semi-synthetic compositions and combinations thereof.

The term “reducing agent” is any compound capable of reducing a compound of the present application to its reduced form. “Reducing agent” includes lipophilic (e.g., lipid-soluble) reducing agents. In one example, the lipid-soluble reducing agent incorporates a hydrophobic moiety, such as a substituted or unsubstituted carbon chain (e.g., a carbon chain consisting of at least 10 carbon atoms). “Reducing agent” also includes hydrophilic (e.g., water-soluble) reducing agents. In one variation, the reducing agent that may be used in the formulation is ubiquinol. In one example, the reducing agent is a “water-soluble reducing agent” when the reducing agent dissolves in water (e.g., at ambient temperature) to produce a clear solution, as opposed to a visibly cloudy, hazy or otherwise inhomogeneous mixture. In one example, the reducing agent is a “water-soluble reducing agent” when it includes at least one (e.g., at least two) hydroxyl group(s) and includes a substituted or unsubstituted linear carbon chain consisting of not more 6, 8, 10, 11, 12, 13, 14 or 15 carbon atoms. An exemplary water-soluble reducing agent is ascorbic acid. The term “water-soluble reducing agent” also includes mixtures of vitamin C with the CBD/THC mixture of the present application. Water-soluble reducing agents can be derivatized to afford an essentially lipid-soluble reducing agent (pro-reducing agent). For example, the water-soluble reducing agent is derivatized with a fatty acid to give, e.g., a fatty acid ester. An exemplary lipid-soluble reducing agent is ascorbic acid-palmitate.

The term “water-soluble” when referring to a formulation or compositions of the present application, means that the formulation when added to an aqueous medium (e.g., water, original beverage) dissolves in the aqueous medium to produce a solution that is essentially clear. In one example, the formulation dissolves in the aqueous medium without heating the resulting mixture above ambient temperature (e.g., 25° C.).

The term “aqueous formulation” refers to a formulation of the present application including at least about 5% (w/w) water. In one example, an aqueous formulation includes at least about 10%, at least about 20%, at least about 30% at least about 40% or at least about 50% (w/w) of water.

The term “pharmaceutical”, “pharmaceutical composition” or pharmaceutical formulation” encompasses “neutraceutical” also referred to as “nutraceutical”), “neutraceutical composition” or “neutraceutical formulation”, respectively. Neutraceutical formulations or neutraceutical compositions may include a pharmaceutically acceptable carrier, such as those described herein.

The term “therapeutically effective amount” is well known in the art and, for example, refers to an amount of a formulation or composition disclosed herein that produces some desired effect at a reasonable benefit/risk ratio applicable to the medical treatment. In certain embodiments, the term refers to that amount necessary or sufficient to eliminate or reduce the medical symptoms for a period of time. The effective amount may vary depending on such factors as the disease or condition being treated, the size of the subject and the severity of the disease or condition. One of ordinary skill in the art may empirically determine the effective amount of a particular composition without undue experimentation.

The term “neutraceutical” or “nutraceutical” is a combination of the terms “nutritional” and “pharmaceutical”. It refers to a composition, which is known or suspected in the art to positively affect human nutrition and/or health.

The term “beverage” describes any water-based liquid, which is suitable for human consumption (i.e., food-grade). A typical beverage of the present application is any “original beverage” in combination with the CBD/THC mixture of the present application. “Original beverage” can be any beverage (e.g., any marketed beverage). The term “original beverage” includes beers, carbonated and non-carbonated waters (e.g., table waters and mineral waters), flavored waters (e.g., fruit-flavored waters), mineralized waters, sports drinks (e.g., Gatorade®), smoothies, neutraceutical drinks, filtered or non-filtered fruit and vegetable juices (e.g., apple juice, orange juice, cranberry juice, pineapple juice, lemonades and combinations thereof) including those juices prepared from concentrates. Exemplary juices include fruit juices having 100% fruit juice (squeezed or made from concentrate), fruit drinks (e.g., 0-29% juice), nectars (e.g., 30-99% juice). The term “original beverage” also includes fruit flavored beverages, carbonated drinks, such as soft-drinks, fruit-flavored carbonates and mixers. Soft drinks include caffeinated soft drinks, such as coke (e.g., Pepsi Cola®, Coca Cola®) and any “diet” versions thereof (e.g., including non-sugar sweeteners). The term “original beverage” also includes teas (e.g., green and black teas, herbal teas) including instant teas, coffee, including instant coffee, chocolate-based drinks, malt-based drinks, milk, drinkable dairy products and beer. The term “original beverage” also includes any liquid or powdered concentrates used to make beverages.

The term “cannabidiol” or “CBD” is a phytocannabinoid that is one of at least 110 active cannabinoids that have been identified in cannabis. CBD may account for up to 40% of the plant's extract, and have been considered to provide a large scope of potential medicinal applications. In another aspect, the term “Cannabinoid” also refers to a compound (such as cannabinol, THC or cannabidiol) that is found in the plant species Cannabis saliva (marijuana), and includes metabolites and synthetic analogues thereof, that may have psychoactive properties. Cannabinoids include compounds, such as THC, that have high affinity for the cannabinoid receptor, and compounds that do not have significant affinity for the cannabinoid receptor, such as cannabidiol (CBD). Cannabinoids also include compounds that have a characteristic dibenzopyran ring structure (such as in THC) and cannabinoids which do not have a pyran ring (such as cannabidiol). Cannabinoids also includes, for example, THC, CBD, dimethyl heptylpentyl cannabidiol (DMHP-CBD) and 6,12-dihydro-6-hydroxy-cannabidiol (see U.S. Pat. Nos. 5,227,537 and 4,876,276); and cannabidiol (−)(CBD) analogs. See Consroe et al., J. Clin. Pharmacol. 21:428S-436S, 1981 and Agurell et al., Pharmacol. Rev. 38:31-43, 1986, which are all incorporated herein by reference. Accordingly, the term “cannabis oil” may comprise of these cannabinoids, including CBD, THC, 11-OH-THC and 11-NOR-9-Carboxy-THC, and other compounds as disclosed herein.

The term “clear beverage” (e.g., clear juice) means any beverage clear (e.g., transparent) to the human eye. Typical clear beverages include carbonated or non-carbonated waters, soft drinks, such as Sprite®, Coke® or root beer, filtered juices and filtered beers. Typical non-clear beverages include orange juice with pulp and milk.

The term “non-alcoholic beverage” includes beverages containing essentially no alcohol. Exemplary non-alcoholic beverages include those listed above for the term “beverage”. The term “non-alcoholic beverage” includes beers, including those generally referred to as “non-alcoholic beers”. In one example, the non-alcoholic beverage includes less than about 10% alcohol by volume. In other examples, the non-alcoholic beverage includes less than about 9%, less than about 8%, less than about 7%, less than about 6% or less than about 5% alcohol by volume.

The term “essentially stable to chemical degradation” refers to the CBD and THC cannabinoid compositions, and mixtures thereof, of the present application as contained in a formulation (e.g., aqueous formulation), beverage or other composition of the present application. In one example, “essentially stable to chemical degradation” means that the composition is stable in its original form and is not converted to another species (e.g., oxidized species or any other species having an essentially different molecular structure), for example, through oxidation, cleavage, rearrangement, polymerization and the like, including those processes induced by light (e.g., radical mechanisms).

The term “essentially clear” is used herein to describe the compositions (e.g., formulations) of the present application. For example, the term “essentially clear” is used to describe an aqueous formulation or a beverage of the present application, such clarity may be assessed by the human eye. In this example, “essentially clear” means that the composition is transparent and essentially free of visible particles and/or precipitation (e.g., not visibly cloudy, hazy or otherwise non-homogeneous). In another example, clarity, haziness or cloudiness of a composition is assessed using light scattering technology, such as dynamic light scattering (DLS), which is useful to measure the sizes of particles, e.g., micelles, contained in a composition. In one example, “essentially clear” means that the median particle size as measured by DLS is less than about 100 nm, the liquid which appears clear to the human eye. In another example, “essentially clear” means that the median particle size is less than about 80 nm; less than about 60 nm; less than about 40 nm; or between about 20 and about 30 nm. For example, in order to prepare a sample (e.g., formulation of the present application) for a DLS measurement, the sample is typically diluted so that the concentration of the solubilizing agent in the diluted sample is between about 1 mM (10⁻³ M) and 0.01 mM (10⁻⁵ M). In another example, the solubilizing agent (e.g., TWEEN-85, TPGS, TPGS-750-M or TPGS-1000) is present in a concentration that is above the critical micelle concentration (CMC) (i.e., the concentration that allows for spontaneous formation of micelles in water). For example, a typical CMC for TPGS in water is about 0.1 to about 0.5 mg/ml.

Alternatively, clarity, haziness or cloudiness of the composition can be determined by measuring the turbidity of the sample, which is useful when the composition is a beverage (e.g., water, soft-drink etc.). In one example, turbidity is measured in FTU (Formazin Turbidity Units) or FNU (Formazin Nephelometric Units), or is measured using a nephelometer. Nephelometric measurements are based on the light-scattering properties of particles. The units of turbidity from a calibrated nephelometer are called Nephelometric Turbidity Units (NTU). In one example, reference standards with known turbidity are used to measure the turbidity of a sample. In one example, a composition of the present application (e.g., a beverage of the present application) is “essentially clear” when the turbidity is not more than about 500% higher than the control (original beverage without an added CBD/THC mixture of the present application, but optionally including a solubilizing agent of the present application). For example, the turbidity of a sample of flavored water is measured to be 2.0 ntu and the turbidity of another sample containing the same flavored water in combination with a fatty acids is measured to be at or below about 8.0 ntu (2.0 ntu+300%=8.0 ntu), then the fatty acids sample is considered to be essentially clear. In another example, a composition of the present application is “essentially clear” when the turbidity is not more than about 300% higher than the control. In yet another example, a composition of the present application is “essentially clear” when the turbidity is not more than about 200%, about 150% or about 100% higher than the control; or is “essentially clear” when the turbidity is not more than about 80%, about 60%, about 40%, about 20% or about 10% higher than the control.

The term “emulsion” as used herein refers to a CBD/THC composition and mixture of the present application emulsified (solubilized) in an aqueous medium using a solubilizing agent of the present application. In one example, the emulsion includes micelles formed between the CBD/THC composition and mixture (i.e., mixture) and the solubilizing agent. When those micelles are sufficiently small, the emulsion is essentially clear. Typically, the emulsion will appear clear to the normal human eye, when those micelles have a median particle size of less than 100 nm. In one example, the micelles in the emulsions of the present application have median particle sizes below 60 nm. In a typical example, micelles formed in an emulsion of the present application have a median particle size between about 20 and about 30 nm. In another example, the emulsion is stable, which means that separation between the aqueous phase and the CBD/THC mixture does essentially not occur (e.g., the emulsion stays clear). A typical aqueous medium, which is used in the emulsions of the present application, is water, which may optionally contain other solubilized molecules, such as salts, coloring agents, flavoring agents and the like. In one example, the aqueous medium of the emulsion does not include an alcoholic solvent, such as ethanol or methanol.

The term “flavonoid” as used herein is recognized in the art. The term “flavonoid” includes those plant pigments found in many foods that are thought to help protect the body from disease (e.g., cancer). These include, for example, epi-gallo catechin gallate (EGCG), epi-gallo catechin (EGC) and epi-catechin (EC).

The term “GRAS” is an acronym for Generally Recognized As Safe as defined under the Federal Food, Drug, and Cosmetic Act, and include any substance that is added to food, subject to approval by FDA, unless the substance is generally recognized, among qualified experts, as having been shown to be safe for its intended use, or unless the use of the substance is excluded from the definition of a food additive.

The term “metal chelator” or “metal chelating moiety” as used herein refers to a compound that combines with a metal ion, such as iron, to form a chelate structure. The chelating agents form coordinate covalent bonds with a metal ion to form the chelates. The term “metal ion” as used herein refers to any physiological, environmental and/or nutritionally relevant metal ion. Such metal ions include certain metal ions such as iron, but may also include lead, mercury and nickel. When EDTA (or disodium EDTA or calcium disodium EDTA) is used in the present application to chelate iron, the chelate forms a Fe³⁺ ethylene-diaminetetraacetic acid (EDTA) complex.

The term “micelle” is used herein according to its art-recognized meaning and includes all forms of micelles, including, for example, spherical micelles, cylindrical micelles, worm-like micelles and sheet-like micelles, and vesicles, formed in water, or mostly water.

The term “pharmaceutically acceptable salts” includes salts of the active compounds which are prepared with relatively nontoxic acids or bases, depending on the particular substituents found on the compounds described herein. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present application contain both basic and acidic functionalities that allow the compounds to be converted into either base or acid addition salts.

The term “THC” (or dronabinol by INN as the pure isomer) or its main isomer (−)-trans-Δ⁹-tetrahydrocannabinol, is the principal psychoactive constituent (or a cannabinoid) of cannabis. THC has been employed effectively for the treatment of anorexia in patients with HIV/AIDS and for refractory nausea and vomiting in patients undergoing chemotherapy. THC is an aromatic terpenoid that has very low solubility in water.

The term “tocopherol” includes all tocopherols, including alpha-, beta-, gamma- and delta tocopherol. The term “tocopherol” also includes tocotrienols.

In another embodiment, the formulation comprises substantially pure CBD/THC mixture, that is greater than 35% pure, greater than 45%, greater than 55%, greater than 65%, greater than 75%, greater than 85%, greater than 90%, greater than 95%, or greater than 98% pure.

These formulations have several advantages. First, they provide a CBD/THC mixture in an essentially clear, aqueous solution. This formulation can enable a consumer to ingest the CBD/THC mixture in a liquid form, for example, in a beverage, such as water. The aqueous formulations are essentially clear, which makes the formulations more appealing to a consumer.

In another aspect, the formulation may include a solubilizing agent described herein, as well as a water-soluble reducing agent (also referred to as a stabilizer). The CBD/THC and their mixture in these formulations (especially aqueous formulations) are stable with respect to chemical degradation (e.g., oxidation). In one example, the chemical stability of the CBD/THC mixture is a result of a synergistic effect between the nature of the solubilizing agent and the water-solubility of the reducing agent (stabilizer): The solubilizing agent is an amphiphilic, nonionic surfactant, which in aqueous solutions allows the CBD/THC mixture to be emulsified in “nanomicelles”, which typically have an average particle size of not more than 150 nm, often below 30 nm. When the CBD/THC mixture is solubilized in the form of these small micelles, a water-soluble (as opposed to lipid-soluble) reducing agent is surprisingly effective in preventing chemical degradation of the CBD/THC mixture in an aqueous solution. For example, the addition of a water-soluble reducing agent diminishes or prevents the degradation of the CBD/THC mixture and extends its average lifetime in solution, for example by at least 5 times.

In another example, the water-soluble reducing agent can be a compound with potential health benefits, such as vitamin C. The combination of two beneficial ingredients (CBD/THC mixture and stabilizer) in a single composition provides greater convenience to a consumer. Another benefit is that the surfactant supplies a nutrient in water (e.g., vitamin E, CoQ10, etc.).

The present application further provides methods of making the formulations. The formulations of the present application can be used in a variety of products, such as foods, beverages, cosmetics and skin-care products (topical application), dietary supplements (e.g., formulated in soft-gelatine capsules) and nutraceuticals. In one embodiment, the present application provides a beverage including a formulation of the present application.

The following table lists exemplary emulsifiers with their HLB values and ranges:

| Emulsifiers/Co-emulsifiers | HLB values (ranges) |
|---|---|
| PEG-2 Hydrogenated Caster Oil | 1.7 |
| Sorbitantrioleate | 1.8 |
| Sorbitantristearate | 2.1 |
| Sorbitan Esters | 2.5-6 |
| Glycerol Stearate | 3.5 |
| Sorbitan Sesquioleate | 3.7 |
| Sorbitan Oleate | 4.3 |
| Sorbitan Monostearate | 4.7 |
| PEG-2 Oleyl Ether | 4.9 |
| PEG-2 Stearyl Ether | 4.9 |
| PEG-7 Hydrogenated Caster oil | 5 |
| PEG-2 Cetyl Ether | 5.3 |
| PEG-4 Sorbitan Stearate | 5.5 |
| PEG-2 Sorbitan Isostearate | 6 |
| Sorbitan Palmitate | 6.7 |
| Phosphatidylcholine | 7.5-10 |
| Sorbitan Monolaurate | 8.6 |
| PEG-40 Sorbitan Peroleate | 9.5 |
| PEG-4 Lauryl Ether | 9.7 |
| Polysorbate 81 | 10 |
| PEG-40 Sorbitan Hexaoleate | 10 |
| PEG-40 Sorbitan Perisostearate | 10 |
| PEG-10 Olive Glycerides | 10 |
| PEG sorbitol Hexaoleate | 10.2 |
| Polysorbate 65 | 10.5 |
| Methylcellulose | 10.7 |
| Gum Arabic | 10-11 |
| Captex 300 | 11 |
| PEG-7 Glyceryl Cocoate | 11 |
| PEG-8 Stearate | 11.1 |
| PEG-400 Monoleate | 11.4 |
| PEG-400 Monostearate | 11.6 |
| Sugar Ester Stearate (S-1170, S-1570, S-1670) | 11-16 |
| PEG-15 Glyceryl Isostearate | 12 |
| PEG-35 Almond Glycerides | 12 |
| PEG-10 Oleyl Ether | 12.4 |
| PEG-8 Isooctylphenyl Ether | 12.4 |
| PEG-10 Stearyl Ether | 12.4 |
| PEG-35 Caster Oil | 12.5 |
| Nonoxynol-9 | 12.9 |
| PEG-10 Cetyl Ether | 12.9 |
| PEG-400 Monolaurate | 13.1 |
| Q-Naturale 200 (Quilaja extract) | 13.5 |
| PEG-40 Hydrogenated Caster Oil | 14 |
| PEG-12 Tridecyl Ether | 14.5 |
| PEG-18 Tridecyl Ether | 14.5 |
| Polysorbate 60 | 14.9 |
| Polysorbate 80 | 15 |
| PEG-20 Glycerol Stearate | 15 |
| Sugar Ester Oleate (OWA-1570) | 15 |
| PEG-20 stearylether | 15.3 |
| Polysorbate 40 | 15.6 |
| Sugar Ester Palmitate (P-1570, P-1670) | 15-16 |
| Sugar Ester Laurate (LWA-1570, L-1695) | 15-16 |
| Polysorbate 20 | 16.7 |
| PEG-60 Hydrogenated Caster Oil | 16 |
| Tocopherol Polyethylene Glycol Succinate | 16-18 |
| (TPGS) | |
| PEG-40 Stearate | 17.3 |
| PEG-50 Stearate | 17.7 |
| Purity Gum Ultra | — |
| Purity Gum BE | — |

In one embodiment, the emulsifiers employed are food grade emulsifiers with high HLB values (>10).

The following abbreviations are used throughout the application: TPGS-polyoxyethanyl-α-tocopheryl succinate (e.g., TPGS-1000, TPGS-600). A number following one of the above abbreviations (e.g., TPGS-600) indicates an average molecular weight of the polyoxyethanyl or poly(ethylene glycol) (PEG) moiety of the compound. A number followed by the abbreviation “Me” or “M” (e.g., TPGS-750-M or TPGS-1000Me) indicates a polyoxyethanyl moiety capped with a methyl group (methoxypolyoxyethanyl or mPEG). Formulations:

An alternative embodiment includes the above ingredients, but may rely on more than one solubilizing agent within any given formulation; i.e., a combination of surfactants (e.g., TPGS, TPGS-1000 or TWEEN-85, in any ratio). In one aspect of the formulation, the solubilizing agent is selected from the group consisting of TPGS (polyoxyethanyl-α-tocopheryl succinate), TPGS-1000 (D-alpha-tocopheryl polyethylene glycol 1000 succinate) and combinations thereof.

In yet another aspect, the solubilizing agent is selected from the group consisting of Poloxamer 188, Polysorbate 80, Polysorbate 20, Vit E-TPGS (TPGS), TPGS-750-M, TPGS-1000, Solutol HS 15, PEG-40 Hydrogenated castor oil (Cremophor RH40), PEG-35 Castor oil (Cremophor EL), PEG-8-glyceryl capylate/caprate (Labrasol), PEG-32-gut may rely nceryl laurate (Gelucire 44/14), PEG-32-glyceryl palmitostearate (Gelucire 50/13); Polysorbate 85, Polyglyceryl-6-dioleate (Caprol MPGO), mixtures of high and low HLB emulsifiers; Sorbitan monooleate (Span 80), Capmul MCM, Maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), Propylene glycol monolaurate (e.g., Capmul PG-12 or Lauroglycol 90), polyglyceryl-3 dioleate (Plurol Oleique CC497), polyglyceryl-3 diisostearate (Plurol Diisostearique) and Lecithin with and without bile salts, or combinations thereof. In another aspect, the water-soluble or water-insoluble reducing agent is selected from the group consisting of L-ascorbic acid-6-palmitate, vitamin C and its salts, alpha, beta, gamma and delta tocopherol or mixtures of tocopherol and alpha, beta, gamma, and delta-tocotrienols or mixtures thereof.

In one aspect of the above formulation, the metal chelator is selected from the group consisting of ethylenediaminetetraacetic acid (EDTA), disodium EDTA and calcium disodium EDTA and mixtures thereof. In another aspect, the bisulfite is sodium bisulfite, potassium bisulfite, sodium metabisulfite or potassium metabisulfite. In another aspect of the formulation, when dissolved in water, provides a solution with a clarity range of about 1,000 to 20 NTU, 100 to 20 NTU, 55 to 35 NTU or about 20 to 35 NTU. In another aspect of the formulation, when dissolved in water, provides a solution that remains stable toward degradation when stored at or below room temperature for a period of at least 6 months or at least 12 months.

In another aspect of the above, the emulsion, when dissolved in water, provides a solution with a clarity range of about 1,000 to 20 NTU, 100 to 20 NTU or about 20 to 35 NTU, and wherein the solution remains stable toward degradation when stored at or below room temperature for a period of at least 6 months or at least 12 months.

In one aspect of the above, there is provided a stabilized beverage, pharmaceutical or nutraceutical product comprising the stabilized powder composition of the above. In one aspect, the stabilized powder composition of the present application, wherein the solution, suited for human consumption is further treated for the inactivation of microbes by a process selected from the group consisting of pasteurization, aseptic packaging, membrane permeation or combinations thereof.

PEG is usually a mixture of oligomers characterized by an average molecular weight. In one example, the PEG has an average molecular weight from about 200 to 5000, from 500 to 1500, from 500 to 800 or about 900 to 1200. In one example, the PEG is PEG-600 or is PEG-750. Both linear and branched PEG moieties can be used as the hydrophilic moiety of the solubilizing agent in the practice of the invention. In one aspect, PEG has between 1000 and 5000 subunits, 1000 subunits, between 100 and 500 subunits, between 10 and 50 subunits, between 1 and 25 subunits, between 15 and 25 subunits, between 5 and 100 subunits or between 1 and 500 subunits.

In one aspect, the ratio of the CBD/THC mixture to the solubilizing agent is from about 1:0.1 (w/w), 1:0.3, or a range of 1:0.3 (w/w) to 1:20 (w/w); or from 1:1 (w/w) to 1:20 (w/w), from 1:1 (w/w) to 1:10 (w/w); from 1:1.3 (w/w) to 1:5 (w/w), from 1:2 (w/w) to 1:4 (w/w), or is about 1:3 (w/w). In another variation, the ratio of the CBD/THC mixture to the solubilizing agent is from about 1:0.1 (w/w) to 1:0.3 (w/w), 1:0.3 (w/w) to 1:1 (w/w), or from 1:0.5 (w/w) to 1:2 (w/w).

### Water-Soluble Reducing Agent:

In one embodiment, the water-soluble reducing agent contained in the formulation (e.g., aqueous formulation) protects the CBD/THC mixture from chemical degradation (e.g., oxidative and/or light-induced processes). For example, addition of vitamin C, a water-soluble vitamin C derivative, or a water-insoluble version of vitamin C to a formulation containing CBD/THC and TPGS serve to prolong the chemical stability of the CBD/THC mixture in the aqueous formulation for at least several weeks. In other embodiments, the water-soluble reducing agent (e.g. based on vitamin C) is added to the formulation in an amount sufficient to both reduce and stabilize the CBD/THC mixture after reduction.

In one example, according to any of the above embodiments, the formulation is an aqueous formulation and includes at least about 5% (w/w) of water, at least 10%, at least 20%, at least 30%, at least 40% or at least 50% (w/w) of water. In another example, the aqueous formulation includes more than 50% (w/w) of water. For example, the aqueous formulation includes at least about 55%, at least 60%, at least 65%, at least 70%, at least 75% or at least 80% (w/w) of water. The aqueous formulation may include more than 80% (w/w) water. For example, the aqueous formulation includes at least about 85%, at least 90%, at least 92%, at least 94% or at least 96% (w/w) of water.

In one embodiment, the aqueous formulation is essentially clear (e.g., free of visible precipitation, cloudiness or haziness). In another example, the CBD/THC mixture of are formulated with TPGS resulting in an aqueous formulation that, likewise, is essentially clear. Clear formulations can be colored. In one example, the formulation is essentially clear when the micelles have a particle size below the visible size (e.g., below 150 nm). The micelles formed by the solubilizing agent containing the CBD/THC mixture have a median (average) particle size of less than about 100 nm. In another example, the micelles formed between the CBD/THC mixture and the solubilizing agent, have a median particle size of less than about 90 nm, less than about 80 nm, less than about 70 nm or less than about 60 nm. In a further example, the micelles formed between the CBD/THC mixture and the solubilizing agent, have a median particle size of less than about 50 nm, less than about 40 nm or less than about 30 nm. In another exemplary embodiment, the average particle size is from about 7 nm to about 90 nm, from about 5 nm to about 70 nm, from about 10 nm to about 50 nm, from about 10 nm to about 30 nm, or from about 7 nm to about 10 nm. In a particular example, the micelles formed between the CBD/THC mixture and the solubilizing agent, have a median particle size between about 30 nm and about 10 nm (e.g., about 25 nm).

### Co-Solvents:

A co-solvent is a substance that is added to a mixture of two or more separate substances that are typically immiscible, in order to make them mixable. Co-solvents are added to increase the solvent power of the primary substance in the mixture. In emulsion systems, co-solvent also mean the substances that are used to attribute to an increase in the solvent capacity of the formulation for incorporated drugs or nutraceuticals and help the dispersion of a system that contains a high proportion of water soluble surfactants.

The popular food grade co-solvents used include ethanol, glycerol, propylene glycol, 1,3-propanediol, butylene glycol, erythritol, xylitol, mannitol, sorbitol, isomalt, polyethylene glycols (PEG)-400, and a combination thereof.

### Vegetable Oils:

Vegetable oils are used to aid the solubility of Cannabis oil into the oil phase of emulsions. The oily fraction used can be selected from the group consisting of Cannabis oil (hemp oil), coconut oil, cottonseed oil, soybean oil, sunflower oil, caster oil, corn oil, olive oil, palm oil, peanut oil, almond oil, sesame oil, rapeseed oil, peppermint oil, canola oil, palm kernel oil, hydrogenated soybean oil, medium-triglyceride, short-chain triglyceride, glyceryl esters of saturated fatty acids, glyceryl behenate, glyceryl distearate, glyceryl isostearate, glyceryl laurate, glyceryl monooleate, glyceryl monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and the combination thereof.

### Flavor Oils:

Flavor oils suitable for preparing the microemulsion of this invention basically used to mask the flavor of cannabis oil and the unpleasant tastes of emulsifiers. They refer to a variety oils that contains one or more volatile compounds. Flavors may be chosen from synthetic flavors, flavoring oils and oil extracts derived from plants, leaves, flowers, fruits and combinations thereof. In this invention, flavor oils are selected from a list of natural cinnamon oil, peppermint oil, clove oil, bay oil, thyme oil, also from artificial, natural or synthetic fruit flavors such as vanilla, chocolate, coffee, cocoa and citrus oil, including lemon, lime, orange, grape, grapefruit, and fruit essences including apple, pear, peach, strawberry, watermelon, raspberry, cherry, plum, pineapple and apricot.

### Stabilizers:

Stabilizers are additives used to help maintain emulsions or prevent degeneration in beverages. Among the most common stabilizers are Acai gum (Gum Arabic), Agar-agar, ammonium alginate, calcium alginate, Carob bean gum (Locust bean gum), Chondrus extract (Carrageenan), ghatti gum, guar gum, pectin, potassium alginate, sodium alginate, sterculia gum (karaya gum), tragacanth, gelatin, lecithin, mono-glycerides, di-glycerides, maltodextrin, xanthan gum, proplylene glycol alginates (PGA), microcrystalline cellulose, sodium carboxymethyl cellulose, Purity Gum 2000 (modified starch), pectin, carrageenan, casein and inulin.

### Anti-Oxidants:

Antioxidants can prevent or slow down the oxidation of bioactive compounds and lipids that are used in beverages. Certain antioxidants, natural or synthetic, include Vitamin A, Vitamin C, Vitamin E (tocopherols), coenzyme Q10, alpha-carotene, astaxanthin, canthaxanthin, cyaniding, quercetin, lutein, lycopene, zeaxanthin, butylated hydroxyanisole (BHA), butylated hydroxytoluene (BHT), propyl gallate, sodium ascorbate, calcium ascorbate, fatty acid esters of ascorbic acid, octyl-gallate, dodecyl gallate, erythrorbic acid, sodium erythorbate, dodecyl gallate, tertiary-butyl hydrochinone (TBHQ), citric acid, 4-hexylresorcinol.

### Chelating Agents:

Chelating agents bind metal ions so that contribute to the stabilization of food color, aroma and texture. Common chelating agents are citric acids, EDTA with its Na- and Ca-salts, oxystearin, orthophosphoric acid, sorbitol, tartaric acid including its Na- and K-salts and thiosulfuric acid with its Na-salt.

In another example, the aqueous formulation does not include an alcoholic solvent, although such inclusion is possible when part of the solubilizing agent (e.g., as in Cremophore, which Contains ethanol). Alcoholic solvents may include solvents, such as ethanol, methanol, propanol, butanol and higher alcohols (e.g., C₅-C₂₀ alcohols). Alcoholic solvents also include polyhydric alcohols, such as ethylene glycol, propylene glycol, glycerol and the like. The term “alcoholic solvent” does not include polymers, such as polymeric versions of the above listed polyhydric alcohols (e.g., poly(alkylene oxides)), such as PEG or PPG).

In one example, according to any of the above embodiments, the concentration of CBD/THC mixture is at least about 20 mg/mL and can be as high as about 60, 80, 100 or more than about 100 mg/mL. In one example, the concentration of CBD/THC mixture in the aqueous formulation of the present application is at least about 1 mg/mL, at least 5 mg/mL, at least 10 mg/mL, at least 20 mg/mL, at least 30 mg/mL, at least 40 mg/mL, at least 50 mg/mL, at least 60 mg/mL, at least 70 mg/mL or at least 80 mg/mL, at least 85 mg/mL, at least 90 mg/mL, at least 95 mg/mL or at least 100 mg/mL, at least 110 mg/mL, at least 120 mg/mL, at least 130 mg/mL, at least 140 mg/mL, at least 50 mg/mL, at least 160 mg/mL, at least 170 mg/mL, at least 180 mg/mL, at least 190 mg/mL or at least about 200 mg/mL. In another example, the concentration of CBD/THC mixture in the aqueous formulation is greater than 200 mg/mL.

In one embodiment, the present application provides a water-soluble formulation comprising bioactive agent or mixtures of bioactive agents as disclosed herein, a water-soluble reducing and/or antioxidizing agent, water-insoluble reducing and/or antioxidizing agent, a solubilizing agent, a metal chelating agent, and a bisulfite salt or a metabisulfite salt.

In particular variations of each of the above aspects and embodiments, the formulation may comprise the CBD/THC mixture and TPGS-1000; natural, non-natural and synthetic surfactants and mixtures of surfactants, including, for example, two or more surfactants of differing structural types (e.g., TPGS-1000 and Tween-80), two or more surfactants from within the same structural class (e.g., TPGS-1000+TPGS-600). In another variation, the formulations may also comprise any of the above combinations as their free alcohols, or as their ether or ester derivatives (of their PEG portion). In another particular variation, the formulations may also comprise antioxidants that are lipophilic in nature (e.g., vitamin C palmitate), hydrophilic in nature (e.g., vitamin C), and any combinations of these, including more than one of each in any formulations. In another particular variation, the formulations may also comprise chelating agents that are lipophilic in nature, hydrophilic in nature (e.g., EDTA, HEDTA, DTPA and NTA), and any combinations of these, and in any number (i.e., more than one of each in any formulation) or ratio. In another particular variation, the formulations may also comprise salts such as salts that are lipophilic in nature (e.g., ammonium salts, such as R₄N⁺X⁻), hydrophilic in nature (e.g., NaHSO₃), and any combinations of these, and in any number (i.e., more than one of each in any formulation) or ratio.

In one example according to any of the above embodiments, the CBD/THC mixture formulation is essentially stable to chemical degradation. In one example, the CBD/THC mixture is essentially stable for at least 30, 60, 180 days, or at least 6 months, 9 months or 12 months, when stored at a temperature below about 25° C. (e.g., 4° C. or 10° C.). Typically, the formulations are stored at about 4° C. At this temperature, the formulations are stable for at least 90 days, at least 6 months or at least 12 months.

Another advantage of the above formulations is that they can be light in color or substantially colorless. The lighter color can be more appealing to the consumer and provides a greater flexibility with respect to the use of coloring agents. In another example, the CBD/THC mixture are emulsified in the formulation in the form of micelles that include the CBD/THC mixture and the solubilizing agent. In a typical emulsion of the present application, the micelles are small in size, and are between about 10 and 30 nm. In another example, the small size of the micelles causes the emulsion to be essentially clear in appearance even at high compound concentrations (e.g., 40, 60, 80 or 100 mg/mL). In one example, the CBD/THC mixture concentration in the aqueous formulations is at least about 20 mg/mL and can be as high as 60, 80, 100 or more than 100 mg/mL.

### Beverages:

In another example, the present application provides a mixture between a formulation of the present application (e.g., a water-soluble formulation) and an original beverage to create a beverage. Exemplary original beverages are described herein and include carbonated or non-carbonated waters, flavored waters, soft drinks and the like. In one example, the mixture (beverage of the present application) includes between about 1 mg/L and about 1000 mg/L of solubilized CBD/THC mixture. In another example, the mixture includes between about 10 mg/L and 500 mg/L of solubilized CBD/THC mixture, between 10 mg/L and 450 mg/mL, between 10 mg/L and 400 mg/mL, between 10 mg/L and 350 mg/mL, between 10 mg/L and 300 mg/mL, or between 10 mg/L and 250 mg/mL of solubilized CBD/THC mixture. In a further example, the mixture includes between about 20 mg/L and about 250 mg/L, between 20 mg/L and 200 mg/mL, between 20 mg/L and 150 mg/mL, between 20 mg/L and 100 mg/mL, or between 20 mg/L and 80 mg/mL, between 20 mg/L and 60 mg/mL, or between 20 mg/L and 40 mg/mL of solubilized CBD/THC mixture. In one aspect, the beverage may comprise of about 1,000 mg or less of solubilized CBD/THC mixture, 500 mg or less, or 250 mg or less of solubilized CBD/THC mixture. In one aspect, the beverage may comprise of a range of about 10 mg to about 500 mg per serving. In another aspect, the beverage may comprise of a range of about 25 mg to about 500 mg per serving. In certain aspects, the beverage may have two servings. In a further example, the beverage further includes a coloring agent and/or a flavoring agent. It is possible to add one or more fruit and/or vegetable juice concentrates and/or flavor improvers to the beverage. For example, a mixture of about LIMETTE citrus (e.g., about 1.38 g/l), cassis (e.g., about 1.04 g/l), mango (e.g., about 1.04 g/l) or combinations thereof, can be added to the beverage. In another example, maltodextrin (e.g., about 20 g/l), fructose (e.g., about 50 g/l) or combinations thereof can be added to the beverage. The finished beverage may be subjected to a primary and, optionally, a secondary filtration.

In yet another example according to any of the above embodiments, the CBD/THC mixture can be solubilized and stabilized in the beverage. For example, the beverage is essentially free of CBD/THC mixture precipitation. The beverage may be essentially clear. Clarity of a beverage can be assessed using turbidity measurements. In one example, the turbidity of the CBD/THC mixture beverage is comparable (e.g., not more than 5 times) of the turbidity of the control beverage. In one example, the turbidity of the beverage is not more than about 500%, not more than 400%, not more than 300% or not more than about 200% higher than the turbidity of the control, not more than about 180%, not more than about 160%, not more than about 140%, not more than about 120% or not more than 100% higher than the turbidity of the control. The turbidity is 100% higher than the control, when the turbidity of the beverage is twice as high as the turbidity of the control.

In another example, the turbidity of the CBD/THC mixture beverage is stable over time. For example, the turbidity of the beverage is stable over a period of at least 60 days, at least 90 days, or at least 180 days when the beverage is stored at ambient temperature (e.g., below about 25° C.). In addition, the beverage can be enriched with vitamins. In one example, the beverage includes at least one B vitamin. Exemplary B-vitamins include vitamin B1, vitamin B2, vitamin B3, vitamin B5, vitamin B6 and vitamin B12. In another example, the beverage includes vitamin E. In one example, the vitamin is first formulated into an aqueous composition, which is subsequently added to the beverage. The solubilizing agent used to solubilize the vitamin can be the same solubilizing agent used to solubilize the CBD/THC mixture.

In one example, the formulation includes from about 0.01% (w/w or wt/wt) to 0.1% (w/w) of CBD/THC mixture, from 0.01% (w/w) to 0.5% (w/w), from 0.01% (w/w) to 1% (w/w), from 0.05% (w/w) to 0.25% (w/w), from 0.1% (w/w) to 1% (w/w), from 0.1% (w/w) to 0.75% (w/w), from 1% (w/w) to 3% (w/w), from 1% (w/w) to 10% (w/w), from 1% (w/w) to 20% (w/w), from 1% (w/w) to 30% (w/w), from 1% (w/w) to 40% (w/w), from 5% to 50% by weight, or from 10% to 30% (w/w), for example, from about 15% to about 25% (w/w).

Solubilizing Agents with a Tocopherol Derivative or a Tocotrienol Derivative:

Methods of making the above solubilizing agents are known in the art as disclosed in U.S. Pat. Nos. 6,045,826, 6,191,172, 6,632,443 and WO 96/17626, all incorporated by reference in their entirety. The soft gel capsules of the present application (based on a soft gel capsule weight of from about 900 mg to about 1200 mg) include a solubilizing agent from about 1% to about 30% by weight, from 5% to 30% (w/w), from 8% to 20% of a solubilizing agent, such as TPGS, TPGS-750-M or TPGS-1000.

### Water-Soluble Reducing Agent:

In another embodiment, the water-soluble reducing agent is vitamin C, a water-soluble vitamin C derivative (e.g., a salt), or a combination thereof. In one embodiment, the compositions are selected from ascorbic acid (vitamin C), a vitamin C derivatives, salts thereof and combinations thereof. In one embodiment, the vitamin C salt, or salt of a vitamin C derivative is an edible (e.g., pharmaceutically acceptable) salt, such as a calcium, sodium, magnesium, potassium and zinc salt. Mixed salts of vitamin C or a vitamin C derivative are also within the scope of the present application. The compositions may include one or more vitamin C derivative. The vitamin C derivative can be any analog of vitamin C. Exemplary vitamin C derivative include those in which at least one of the hydroxyl groups of the ascorbic acid molecule (e.g., 2-OH, 3-OH, 5-OH, 6-OH) is derivatized with a modifying group (see e.g., U.S. Pat. No. 5,078,989). In another embodiment, the compositions may include vitamin C as well as at least one vitamin C derivative.

In another embodiment, the stabilizer is in excess in relation to the CBD/THC mixture, or the CBD/THC mixture is in excess of the stabilizer. In another exemplary embodiment, the ratio of the CBD/THC mixture to the stabilizer is from about 1:1 (w/w) to about 1:6 (w/w), from 1:1 (w/w) to 1:5 (w/w), from 1:1.3 (w/w) to 1:3 (w/w), from 1:2 (w/w) to 1:4 (w/w), or about 1:3 (w/w). In another embodiment, the ratio of the stabilizer to the CBD/THC mixture is from about 1:1 (w/w) to about 1:6 (w/w), from 1:1 (w/w) to 1:5 (w/w), from 1:1.3 (w/w) to 1:3 (w/w), from 1:2 (w/w) to 1:4 (w/w) or about 1:3 (w/w).

In another embodiment, the stabilizer is vitamin C or a vitamin C derivative. In one example, the vitamin C or the vitamin C derivative is used in a molar excess in relation to the CBD/THC mixture. In another exemplary embodiment, the ratio of the CBD/THC mixture to vitamin C or vitamin C derivative is from about 1:1 (w/w) to 1:6 (w/w), from 1:1 (w/w) to 1:10 (w/w), from 1:1.3 (w/w) to 1:5 (w/w), from 1:2 (w/w) to 1:4 (w/w), or about 1:3 (w/w).

### The Metal Chelating Agent:

In another embodiment, the metal chelator, chelating agent or metal chelating moiety is a chelator that has demonstrated affinity metal ions. Such metal ions include iron, but may also include lead, mercury and nickel. In one aspect, the chelator is EDTA or ethylenediaminetetraacetic acid disodium salt dihydrate and the metal ion is iron (II) or iron (III). In one aspect, the metal ion is iron (III). In one embodiment, the formulations of the present application include from about 0.001% to about 0.01% by weight of the chelator relative to the CBD/THC mixture (w/w), from 0.01% to 0.1%, from 0.1% to 0.5%, from 0.5% to 1.0%, from 1.0% to 2.0%, from 2.0% to 4.0%, from 4.0% to 6.0%, or about 4% of the chelator relative to the CBD/THC mixture. In another embodiment, the formulations of the present application include from about 6.0% to about 10.0% by weight of the chelator relative to the CBD/THC mixture (w/w), from 10.0% to about 15%, or from 15% to about 20% by weight of the chelator relative to the CBD/THC mixture.

### Other Components:

The formulations described herein (either aqueous or non-aqueous) can further include ingredients useful to stabilize the composition, promote the bioavailability of the CBD/THC mixture. Additives of the present formulations may include one or more alternative solubilizing agents, pharmaceutical drug molecules, antibiotics, sterols, vitamins, provitamins, carotenoids (e.g., alpha and beta-carotenes, cryptoxanthin, lutein and zeaxanthin), phospholipids, L-carnitine, starches, sugars, fats, stabilizers, reducing agents, free radical scavengers, amino acids, amino acid analogs, proteins, solvents, emulsifiers, adjuvants, sweeteners, fillers, flavoring agents, coloring agents, lubricants, binders, moisturizing agents, preservatives, suspending agents, starch, hydrolyzed starch(es), derivatives thereof and combinations thereof.

In one embodiment, the formulation may further comprise gelatin, sorbitol, glycerin or any ester derivatives therefrom. In another embodiment, the formulation further comprises polysorbate 80, hydroxylated lecitin, medium chain triglycerides, annato seed extract, rice bran oil, carotenoids, titanium dioxide, suspending agents such as silica (silicon dioxide), riboflavin or mixtures thereof. Other additives can be incorporated into the present formulations including phospholipids, L-carnitine, starches, sugars, fats, stabilizers, amino acids, proteins, flavorings, coloring agents, hydrolyzed starch(es) or combinations thereof.

Vitamin(s) in a unit dosage form of the present application are present in amount ranging from about 5 mg to about 500 mg, 10 mg to 400 mg or from about 250 mg to 400 mg. Most specifically, the vitamin(s) is present in an amount ranging from about 10 mg to 50 mg. For example, B vitamins are in usually incorporated in the range of about 1 milligram to 10 milligrams, i.e., from about 3 micrograms to 50 micrograms of B12. Folic acid, for example, is generally incorporated in a range of about 50 to 400 micrograms, biotin is generally incorporated in a range of about 25 to 700 micrograms and cyanocobalamin is incorporated in a range of about 3 micrograms to 50 micrograms.

Mineral(s) in a unit dosage form of the present application are present in an amount ranging from about 25 mg to about 1000 mg, from about 25 mg to about 500 mg, or from about 100 mg to about 600 mg. In the formulations, the additional components are usually a minor component (from 0.001% to 20% by weight or preferably from 0.01% to 10% by weight) with the remainder being various vehicles or carriers and processing aids helpful for forming the desired dosing form.

### Pharmaceutical Formulations:

The present application provides pharmaceutical formulations comprising a formulation of the present application and a pharmaceutically acceptable carrier. Pharmaceutical formulations include nutraceutical formulations. An exemplary unit dosage form (e.g., contained in a soft gel capsule) includes a pharmaceutical grade CBD/THC mixture in an amount of about 1% to about 30% by weight; from about 3% to about 20% (w/w), or from about 5% to about 20% of a CBD/THC mixture. Typically, soft-gel formulations include from about 5% to about 30% (w/w) of CBD/THC mixture, from about 15% to about 40% (w/w) solubilizing agent (e.g., TPGS or TPGS-1000), from 30% to 60% (w/w) lipophilic carrier (e.g., fish oil) and from 1% to 10% (w/w) viscosity enhancer (e.g., beeswax). In another embodiment, the soft gel capsule of the present application includes CBD/THC mixture, vitamin C, solubilizing agent (e.g., TPGS or TPGS-1000), beeswax and a lipophilic carrier (e.g., fish oil). In another embodiment, the CBD/THC mixture are combined with a solubilizing agent useful to improve the bioavailability of the CBD/THC mixture. Such formulations may further contain additional active ingredients and/or pharmaceutically or cosmetically acceptable additives or vehicles, including solvents, adjuvants, excipients, sweeteners, fillers, colorants, flavoring agents, lubricants, binders, moisturizing agents, preservatives and mixtures thereof.

The pharmaceutical composition can be prepared according to known methods. Formulations are described in detail in a number of sources, which are well known and readily available. For example, Remington's Pharmaceutical Science by E. W. Martin describes such formulations. The compositions of the subject present application are formulated such that an effective amount of the CBD/THC mixture is provided in the composition. Pharmaceutical compositions are provided which comprise, an active ingredient as described above, and an effective amount of one or more pharmaceutically acceptable excipients, vehicles, carriers or diluents. Examples of such carriers include ethanol, dimethyl sulfoxide, glycerol, silica, alumina, starch, and equivalent carriers and diluents. Acceptable carriers can be either solid or liquid. Solid form preparations include powders, tablets, pills, capsules, cachets, suppositories and dispersible granules. A solid carrier can be one or more substances, which may act as diluents, flavoring agents, solubilizing agents, lubricants, suspending agents, binders, preservatives, tablet disintegrating agents or encapsulating materials.

For oral administration, the pharmaceutical compositions can take the form of tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in conventional manner.

The disclosed pharmaceutical compositions can be subdivided into unit doses containing appropriate quantities of the active component. The unit dosage form can be a packaged preparation, such as packeted tablets, capsules, and powders in paper or plastic containers or in vials or ampoules. The unit dosage can be a liquid based preparation or formulated to be incorporated into solid food products, chewing gum, or lozenges. Pharmaceutically acceptable salts (counter ions) can be prepared by ion-exchange chromatography or other methods as are well known in the art. The formulations of the present application may be adapted to the route of administration. Those skilled in the art will recognize various synthetic methodologies that may be employed to prepare non-toxic pharmaceutical formulations incorporating the compounds described herein. A wide variety of non-toxic pharmaceutically acceptable solvents that may be used to prepare solvates of the compounds of the present application, such as water, ethanol, propylene glycol, mineral oil, vegetable oil and dimethylsulfoxide (DMSO).

The compositions of the present application may be administered orally, topically, parenterally or rectally in dosage unit formulations containing conventional pharmaceutically acceptable carriers, adjuvants and vehicles. The best method of administration may be a combination of methods. The term parenteral as used includes subcutaneous injections, intradermal, intravascular (e.g., intravenous), intramuscular, spinal, intrathecal injection or like injection or infusion techniques. The formulations are in a form suitable for oral use, such as tablets, troches, lozenges, aqueous or oily suspensions, dispersible powders or granules, emulsion, hard or soft capsules, soft gel capsules, or syrups or elixirs. The formulations may be prepared according to any method known in the art for the manufacture of pharmaceutical formulations and nutraceuticals, and such compositions may contain one or more agents selected from the group consisting of sweetening agents, flavoring agents, coloring agents and preserving agents in order to provide pharmaceutically palatable preparations. Tablets may contain the active ingredient in admixture with non-toxic pharmaceutically acceptable excipients that are suitable for the manufacture of tablets. These excipients may be inert diluents, such as calcium carbonate, sodium carbonate, lactose, calcium phosphate or sodium phosphate; granulating and disintegrating agents, such as corn starch, or alginic acid; binding agents, such as starch, gelatin or acacia; and lubricating agents, such as magnesium stearate, stearic acid or talc. The tablets may be uncoated or they may be coated by known techniques to delay disintegration and absorption in the gastrointestinal tract and thereby provide a sustained action over a longer period. A time delay material such as glyceryl monostearate or glyceryl distearate may be employed. Formulations for oral use may also be hard gelatin capsules wherein the active ingredient is mixed with an inert solid diluent, such as calcium carbonate, calcium phosphate or kaolin, or as soft gelatin capsules wherein the active ingredient is mixed with water or an oil medium, such as peanut oil, liquid paraffin or olive oil. Aqueous suspensions contain the active materials in admixture with excipients for the manufacture of aqueous suspensions. Such excipients are suspending agents, such as sodium carboxymethylcellulose, methylcellulose, hydroxypropylmethyl cellulose, sodium alginate, polyvinylpyrrolidone, gum tragacanth and gum acacia; and dispersing or wetting agents, which may be a naturally-occurring phosphatide, such as lecithin, or condensation products of an alkylene oxide with fatty acids, such as polyoxyethylene stearate, or condensation products of ethylene oxide with long chain aliphatic alcohols, such as heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, such as beeswax, hard paraffin or cetyl alcohol. Sweetening agents and flavoring agents may be added to provide palatable oral preparations. These compositions may be preserved by the addition of an anti-oxidant such as ascorbic acid. Dispersible powders and granules suitable for preparation of an aqueous suspension by the addition of water provide the active ingredient in admixture with a dispersing or wetting agent, suspending agent and one or more preservatives. Additional excipients, for example sweetening, flavoring and coloring agents, may also be present.

In one embodiment, the formulations may also be in the form of oil-in-water emulsions and water-in-oil emulsions. The oily phase may be a vegetable oil, such as olive oil or arachis oil, or a mineral oil, such as liquid paraffin or mixtures of these. Suitable emulsifying agents may be naturally-occurring gums, such as gum acacia or gum tragacanth; naturally-occurring phosphatides, such as soy bean, lecithin, and esters or partial esters derived from fatty acids and hexitol; anhydrides, such as sorbitan monooleate; and condensation products of the partial esters with ethylene oxide, such as polyoxyethylene sorbitan monooleate. The emulsions may also contain sweetening and flavoring agents. Syrups and elixirs may be formulated with sweetening agents, for example glycerol, propylene glycol, sorbitol or sucrose. The formulations may also contain a demulcent, a preservative and flavoring and coloring agents. The formulations may be in the form of a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, for example as a solution in 1,3-butanediol. The acceptable vehicles and solvents include water, Ringer's solution and isotonic sodium chloride solution. Sterile, fixed oils may be employed as a solvent or suspending medium. Any bland fixed oil may be employed including synthetic mono- or diglycerides; or fatty acids such as oleic acid for preparation of injectables.

For administration to non-human animals, the formulations of the present application may be added to the animal's feed or drinking water. It may be formulated for animal feed and drinking water products so that the animal takes in an appropriate quantity of the compound in its diet. The compound may be a composition as a premix for addition to the feed or drinking water. The composition can also be added as a food or drink supplement for humans. Dosage levels (with respect to CBD/THC mixture or composition) of the order of from about 1 mg to about 250 mg per kilogram of body weight per day are useful. For example, a dosage level from about 25 mg to about 150 mg per kilogram of body weight per day, are useful. The amount of active ingredient that may be combined with the carrier materials to produce a single dosage form will vary depending upon the condition being treated and the particular mode of administration. Dosage unit forms will generally contain between from about 1 mg to about 500 mg of the CBD/THC mixture and carotenoids (e.g., astaxanthin, fucoxanthin, cantaxanthin and the like). For example, dosage unit forms of about 1 mg to 250 mg, 1 mg to 100 mg or 1 mg to about 80, 60, 40, 20 or 10 mg are useful. Frequency of dosage may also vary depending on the compound used and the particular disease treated. For treatment of most disorders, a dosage regimen of 4 times daily or less may be used. The specific dose level for any particular patient will depend upon a variety of factors including the activity of the specific compound employed, the age, body weight, general health, sex, diet, time of administration, route of administration and rate of excretion, drug combination and the severity of the particular disease undergoing therapy. Also provided are packaged formulations and instructions for use of the tablet, capsule, soft gel capsule, elixir, etc. Typically, the dosage requirement is between about 1 to 4 dosages a day.

### Methods of Making the Exemplary Formulations:

The present application also provides methods (e.g., processes) of making the formulations and compositions of the present application.

### Materials and Methods:

### Oils and Chemicals:

CBD and THC oil were purchased in local dispensaries. CBD oils Botanacor Elixinol AMBR-25-2 and Folium Biosciences Phytocannabinoid Rich Hemp Oil were purchased in Denver, Colo. THC oils Indica CO₂ oil syringe and Mountain mix sativa wax PHO/BPO wax were purchased in Denver, Colo.

The three Outco THC oils were purchased in California.

Kentucky oil #1 was purchased from Atalo Holdings, Kentucky. CBD oil #9 was purchased in Denver, Colo. NAV-WeedMD CBD/THC oil 37-35 and NAV-WeedMD CBD/THC oil 49-38 was purchased from WeedMD, Inc., Aylmer, Ontario.

Captex 300 EP/NF was obtained from ABITEC Corporation, Columbus, Ohio. Polysorbate 40 was obtained from Spektrum Chemical, New Brunswick, N.J.

Q-Naturale 200 and Purity Gum Ultra were obtained from Ingredion INC., Bridgewater, N.J.

Gum arabic was obtained from TIC Gums, Belcamp, Md.

Polysorbate 80, glycerol, PEG 400 mono laureate, propylene glycol, mannitol, polysorbate 60, ethanol, castor oil, PEG 40 stearate, methyl cellulose, canola oil, sunflower oil, polysorbate 20, coconut oil, peppermint oil, propylene glycol alginates (PGA) were obtained from Universal Preserv-A-Chem Inc., Sommerset, N.J.

Sugar Ester Stearate S-1170, Sugar Ester Oleate (OWA-1570) and Sugar Ester Laurate LWA-1570 were obtained from Mitsubishi-Kagaku Foods Corporation, Singapore.

Sorbitan Palmitate was obtained from Universal Preserv-A-Chem Inc., Sommerset, N.J.

PEG-8 Di-Stearate was obtained from Universal Preserv-A-Chem Inc., Sommerset, N.J.

Glycerol Stearate was obtained from Best of Chemicals, Shirley, N.Y.

### Preparation of Emulsions:

The components for each emulsion (according to Table 1) were weighed into a 50 ml Erlenmeyer flask for a 15 g batch. The flask was placed into an oil bath with a temperature range of 65 to 95° C. for 10 minutes. Stirring was done with a magnetic stirrer. The flask then is cooled to room temperature in an ice bath.

### Turbidity Measurements:

Turbidity of the emissions was measured with an Oakton T-100 Turbidity meter form OAKTON Instruments, Vernon Hills, US.

For Botanacor Elixinol AMBR-25-2, which Contains 33.82% CBD.

### Example 1

Emulsifier to CBD Oil Ratio: 4.75:1

| | | | | |
|---|---|---|---|---|
| Botanacor Elixinol AMBR-25-2 | 1.2 | g | 8.00% | 2.71% pure CBD |
| Captex 300 EP/NF | 2.5 | g | 16.7% | |
| Polysorbate 40 | 1.5 | g | 10.0% | |
| Q-Naturale 200 | 1.7 | g | 11.3% | |
| Distilled water | 8.1 | g | 54.0% | |
| Subtotal | 15 | g | 100% | |

### Example 2

Emulsifier to CBD Oil Ratio: 3.67:1

| | | | | |
|---|---|---|---|---|
| Botanacor Elixinol AMBR-25-2 | 1.5 | g | 10% | 3.38% pure CBD |
| Sunflower oil | 0.5 | g | 3.33% | |
| Captex 300 EP/NF | 2.5 | g | 16.7% | |
| Polysorbate 80 | 1.5 | g | 10.0% | |
| Q-Naturale 200 | 1.5 | g | 10.0% | |
| Glycerol | 0.5 | g | 3.33% | |
| Distilled water | 7.0 | g | 46.7% | |
| Subtotal | 15 | g | 100% | |

### Example 3

Emulsifier to CBD Oil Ratio: 2:1

| | | | | |
|---|---|---|---|---|
| Botanacor Elixinol AMBR-25-2 | 1.5 | g | 10% | 3.38% pure CBD |
| PEG 400 Monolaurate | 1.0 | g | 6.7% | |
| Purity Gum Ultra | 1.5 | g | 10% | |
| Glycerol Stearate | 0.8 | g | 5.3% | |
| Propylene Glycol | 1.5 | g | 10.0% | |
| Mannitol | 0.2 | g | 1.3% | |
| Glycerol | 0.5 | g | 3.3% | |
| Distilled water | 8.0 | g | 53.3% | |
| Subtotal | 15 | g | 100% | |

For Folium Biosciences Phytocannabinoid Rich Hemp Oil, which Contains 68.5% CBD

### Example 4

Emulsifier to CBD Oil Ratio 11.4:1

| | | | | |
|---|---|---|---|---|
| Folium Biosciences | 0.5 | g | 3.3% | 2.28% pure CBD |
| Phytocannabinoid | | | | |
| Rich Hemp Oil | | | | |
| Q-Naturale 200 | 1.5 | g | 10% | |
| Polysorbate 60 | 1.5 | g | 10% | |
| Sugar Ester Stearate S-1170 | 1.0 | g | 6.7% | |
| PEG 40 Stearate | 1.2 | g | 8.0% | |
| Vitamin E TPGS | 0.5 | g | 3.3% | |
| Glycerol | 1.0 | g | 6.7% | |
| Distilled water | 7.8 | g | 52.0% | |
| Subtotal | 15 | g | 100% | |

### Example 5

Emulsifier to CBD Oil Ratio 7:1

| | | | | |
|---|---|---|---|---|
| Folium Biosciences | 1.0 | g | 6.7% | 4.57% Pure CBD |
| Phytocannabinoid | | | | |
| Rich Hemp Oil | | | | |
| Q-Naturale 200 | 1.5 | g | 10% | |
| Polysorbate 80 | 2.5 | g | 16.7% | |
| Polysorbate 60 | 1.5 | g | 10.0% | |
| Sugar Ester OleateOWA-1570 | 0.9 | g | 6.00% | |
| Purity Gum Ultra | 0.6 | g | 4.00% | |
| Glycerol | 0.5 | g | 3.3% | |
| Ethanol | 0.5 | g | 3.3% | |
| Distilled water | 6.0 | g | 40.0% | |
| Subtotal | 15 | g | 100% | |

### Example 6

Emulsifier to CBD Oil Ratio 4.5:1

| | | | | |
|---|---|---|---|---|
| Folium Biosciences | 1.0 | g | 6.7% | 4.57% Pure CBD |
| Phytocannabinoid | | | | |
| Rich Hemp Oil | | | | |
| Hemp oil | 1.0 | g | 6.7% | |
| Polysorbate 80 | 2.5 | g | 16.7% | |
| Sugar Ester OleateOWA-1570 | 1.1 | g | 7.3% | |
| Sorbitan Palmitate | 0.9 | g | 6.0% | |
| Propylene Glycol | 1.0 | g | 6.7% | |
| Distilled water | 7.5 | g | 50.0% | |
| Subtotal | 15 | g | 100% | |

### Example 7

Emulsifier to CBD Oil Ratio 4.58:1

| | | | | |
|---|---|---|---|---|
| Kentucky CBD oil #1 | 1.2 | g | 8.00% | 2.82% Pure CBD |
| Castor oil | 0.5 | g | 3.33% | |
| Q-Naturale 200 | 2.5 | g | 16.7% | |
| PEG 40 Stearate | 1.5 | g | 10.0% | |
| Polysorbate 60 | 1.5 | g | 10.0% | |
| Propylene Glycol | 0.5 | g | 3.33% | |
| Distilled water | 7.3 | g | 48.7% | |
| Subtotal | 15 | g | 100% | |

### Example 8

Emulsifier to CBD Oil Ratio 2.92:1

| | | | | |
|---|---|---|---|---|
| Kentucky CBD Oil #1 | 1.2 | g | 8.00% | 2.82% Pure CBD |
| Q-Naturale 200 | 2.0 | g | 13.3% | |
| Polysorbate 80 | 1.0 | g | 6.7% | |
| Methylcellulose | 0.2 | g | 1.33% | |
| Sorbitan Monostearate | 0.3 | g | 2.00% | |
| Gum Arabic | 0.1 | g | 0.67% | |
| Distilled water | 10.2 | g | 68.0% | |
| Subtotal | 15 | g | 100% | |

For Colorado Denver CBD Oil, which Contains 64.9% CBD Oil

### Example 9

Emulsifier to CBD Oil Ratio 4.3:1

| | | | | |
|---|---|---|---|---|
| Denver CBD Oil | 1.0 | g | 6.67% | 4.33% Pure CBD |
| Canola oil | 0.5 | g | 3.33% | |
| Polysorbate 60 | 2.7 | g | 18.0% | |
| Purity Gum Ultra | 0.8 | g | 5.33% | |
| Q-Naturale 200 | 0.8 | g | 5.33% | |
| Distilled water | 9.2 | g | 61.3% | |
| Subtotal | 15 | g | 100% | |

### Example 10

Emulsifier to CBD Oil Ratio 4:1

| | | | | |
|---|---|---|---|---|
| Denver CBD Oil | 1.0 | g | 6.67% | 4.33% Pure CBD |
| Canola oil | 0.5 | g | 3.33% | |
| Polysorbate 80 | 2.0 | g | 13.3% | |
| Purity Gum Ultra | 1.0 | g | 6.67% | |
| Q-Naturale 200 | 1.0 | g | 6.67% | |
| Propylene Glycol | 0.8 | g | 5.33% | |
| Distilled water | 8.7 | g | 58.0% | |
| Subtotal | 15 | g | 100% | |

For Indica CO2 Oil Syringe THC Oil, which Contains 77.8% THC

### Example 11

Emulsifier to THC Oil Ratio 4.7:1

| | | | | |
|---|---|---|---|---|
| Indica CO2 oil syringe | 1.0 | g | 6.7% | 5.19% |
| THC oil | | | | |
| Sunflower oil | 0.5 | g | 3.3% | |
| Vanilla flavor oil | 0.3 | g | 2.0% | |
| Captex 300 EP/NF | 1.7 | g | 11% | |
| Polysorbate 40 | 1.5 | g | 10% | |
| Vitamin E TPGS | 1.5 | g | 10% | |
| Propylene Glycol | 1.0 | g | 6.7% | |
| Glycerol | 0.5 | g | 3.3% | |
| Distilled water | 7.0 | g | 47% | |
| Subtotal | 15 | g | 100% | |

### Example 12

Emulsifier to THC Oil Ratio 2.67:1, at 4.67%

| | | | |
|---|---|---|---|
| Indica CO2 oil syringe | 0.9 | g | 6.00% |
| THC oil | | | |
| Polysorbate 20 | 1.2 | g | 8.00% |
| PEG 40 Stearate | 0.6 | g | 4.00% |
| Sugar Ester Oleate (OWA-1570) | 0.6 | g | 4.00% |
| Erythritol | 0.3 | g | 2.00% |
| Distilled water | 11 | g | 76.0% |
| Subtotal | 15 | g | 100% |

For Mountain Mix Sativa Wax PHO/BPO Wax, which Contains 77.9% THC

### Example 13

Emulsifier to THC Oil Ratio 2.92:1

| | | | |
|---|---|---|---|
| Mountain mix sativa wax | 1.2 g | 8.0% | 6.23% pure THC |
| PHO/BPO wax | | | |
| Coconut oil | 0.5 g | 3.3% | |
| Polysorbate 60 | 1.2 g | 8.0% | |
| Purity Gum Ultra | 0.9 g | 6.0% | |
| PEG 40 Stearate | 1.0 g | 6.7% | |
| Methylcellulose | 0.4 g | 2.7% | |
| Glycerol | 0.5 g | 3.3% | |
| Ethanol | 1.2 g | 8.0% | |
| Distilled water | 8.1 g | 54% | |

For Outco 17BG0018 F2, which Contains 86.8%

### Example 14

Emulsifier to THC Oil Ratio 3.67:1

| | | | | |
|---|---|---|---|---|
| Outco 17BG0018 F2 | 1.2 | g | 8.00% | 6.94% Pure THC |
| Hemp oil | 0.5 | g | 3.33% | |
| Peppermint oil | 0.3 | g | 2.00% | |
| Cremophor RH 40 | 1.5 | g | 10.0% | |
| Polysorbate 60 | 1.9 | g | 12.7% | |
| Purity Gum BE | 1.0 | g | 6.67% | |
| Glycerol | 1.0 | g | 6.67% | |
| Mixed Tocopherols | 0.3 | g | 2.00% | |
| Distilled water | 7.3 | g | 48.7% | |
| Subtotal | 15 | g | 100% | |

### Example 15

Emulsifier to THC Oil Ratio 2.47:1

| | | | | |
|---|---|---|---|---|
| Outco 17BG0018 F2 | 1.5 | g | 10.0% | 8.68% Pure THC |
| Hemp oil | 0.5 | g | 3.33% | |
| Peppermint oil | 0.3 | g | 2.00% | |
| Polysorbate 80 | 1.5 | g | 10.0% | |
| Purity Gum Ultra | 1.2 | g | 8.00% | |
| Q-Naturale 200 | 1.0 | g | 6.67% | |
| Propylene Glycol | 0.5 | g | 3.33% | |
| Mixed Tocopherols | 0.3 | g | 2.00% | |
| Distilled water | 8.2 | g | 54.7% | |
| Subtotal | 15 | g | 100% | |

For Outco Space Bomb Sativa Vape, which Contains 67.0% THC Oil

### Example 16

Emulsifier to THC Oil Ratio 3.6:1

| | | | | |
|---|---|---|---|---|
| Outco Space Bomb Sativa | 1.0 | g | 6.67% | 4.47% Pure THC |
| Vape | | | | |
| Canola oil | 0.5 | g | 3.33% | |
| Polysorbate 80 | 1.5 | g | 10.0% | |
| Q-Naturale 200 | 1.2 | g | 8.00% | |
| Purity Gum Ultra | 0.9 | g | 6.00% | |
| Propylene Glycol | 0.4 | g | 2.67% | |
| Mixed Tocopherol | 0.3 | g | 2.00% | |
| Distilled water | 9.2 | g | 61.3% | |
| Subtotal | 15 | g | 100% | |

### Example 17

Emulsifier to THC Oil Ratio 2.6:1

| | | | | |
|---|---|---|---|---|
| Outco Space Bomb Sativa | 1.0 | g | 6.67% | 4.47% Pure THC |
| Vape | | | | |
| Canola oil | 0.5 | g | 3.33% | |
| Polysorbate 60 | 1.2 | g | 8.00% | |
| Q-Naturale 200 | 0.8 | g | 5.33% | |
| Sugar Ester LaurateLWA- | 0.6 | g | 4.00% | |
| 1570 | | | | |
| Propylene Glycol | 0.5 | g | 3.33% | |
| Mixed Tocopherol | 0.3 | g | 2.00% | |
| proplylene glycol alginates | 0.15 | g | 1.00% | |
| (PGA) | | | | |
| Distilled water | 9.95 | g | 66.3% | |
| Subtotal | 15 | g | 100% | |

### Example 18

Emulsifier to THC Oil Ratio 2:1

| | | | | |
|---|---|---|---|---|
| Outco Space Bomb Sativa | 1.5 | g | 10.00% | 6.70% Pure THC |
| Vape | | | | |
| Canola oil | 0.5 | g | 3.33% | |
| Vitamin E TPGS | 1.5 | g | 10.0% | |
| Polysorbate 60 | 0.9 | g | 6.00% | |
| Purity Gum Ultra | 0.6 | g | 4.00% | |
| EDTA | 0.3 | g | 2.00% | |
| proplylene glycol alginates | 0.15 | g | 1.00% | |
| (PGA) | | | | |
| Distilled water | 9.55 | g | 63.7% | |
| Subtotal | 15 | g | 100% | |

For Outco 17BG0017 F2, which Contains 86.0% THC

### Example 19

Emulsifier to THC Oil Ratio 4.1:1

| | | | | |
|---|---|---|---|---|
| Outco 17BG0017 F2 | 1.0 | g | 6.67% | 5.74% Pure THC |
| Castor oil | 0.5 | g | 3.33% | |
| Peppermint oil | 0.5 | g | 3.33% | |
| Polysorbate 80 | 1.5 | g | 10.0% | |
| Vitamin E TPGS | 1.2 | g | 8.00% | |
| Purity Gum Ultra | 0.9 | g | 6.00% | |
| Propylene Glycol | 0.6 | g | 4.00% | |
| Mixed Tocopherols | 0.3 | g | 2.00% | |
| Distilled water | 8.5 | g | 56.7% | |
| Subtotal | 15 | g | 100% | |

### Example 20

Emulsifier to THC Oil Ratio 2.6:1

| | | | | |
|---|---|---|---|---|
| Outco 17BG0017 F2 | 1.0 | g | 6.67% | 5.74% Pure THC |
| Castor oil | 0.5 | g | 3.33% | |
| Peppermint oil | 0.3 | g | 2.00% | |
| Polysorbate 80 | 1.2 | g | 8.00% | |
| Vitamin E TPGS | 0.8 | g | 6.77% | |
| Purity Gum Ultra | 0.6 | g | 4.00% | |
| Propylene Glycol | 0.6 | g | 4.00% | |
| Mixed Tocopherols | 0.3 | g | 2.00% | |
| EDTA | 0.2 | g | 1.33% | |
| Distilled water | 9.5 | g | 63.3% | |
| Subtotal | 15 | g | 100% | |

### Example 21

Emulsifier to THC Oil Ratio 1.5:1

| | | | | |
|---|---|---|---|---|
| Outco 17BG0017 F2 | 1.0 | g | 6.67% | 5.74% THC |
| PEG-8 Stearate | 1.5 | g | 10.0% | |
| Distilled water | 12.5 | g | 83.3% | |
| Subtotal | 15 | g | 100% | |

### Example 22

Emulsifier to CBD/THC Oil Ratio 4.5:1

| | | | | |
|---|---|---|---|---|
| NAV-WeedMD | 7.7 | g | 7.00% | 3.44% total CBD |
| CBD/THC oil 49-38 | | | | and 2.69% total |
| | | | | THC |
| Canola Oil | 4.7 | g | 4.27% | |
| Cremophor RH40 | 33.1 | g | 30.1% | |
| Vitamin E TPGS | 1.7 | g | 1.55% | |
| Glycerol | 5.0 | g | 4.55% | |
| Ascorbic Acid | 0.2 | g | 0.18% | |
| Mixed Tocopherols | 1.4 | g | 1.27% | |
| Distilled water | 56.2 | g | 51.1% | |
| Subtotal | 110 | g | 100% | |

### Example 23

Emulsifier to CBD/THC Oil Ratio 4.55:1

| | | | | |
|---|---|---|---|---|
| NAV-WeedMD | 6.6 | g | 6.60% | 3.25% total CBD |
| CBD/THC oil 49-38 | | | | and 2.53% total |
| | | | | THC |
| Corn oil | 4.5 | g | 4.5% | |
| Clove oil | 0.5 | g | 0.5% | |
| Vitamin E TPGS | 27 | g | 27% | |
| Capmul MCM | 3.0 | g | 3.0% | |
| Ascorbic Acid | 0.2 | g | 0.2% | |
| Mixed Tocopherols | 1.5 | g | 1.5% | |
| Distilled water | 57 | g | 57% | |
| Subtotal | 100 | g | 100% | |

### Example 24

Emulsifier to CBD/THC Oil Ratio 4.0:1

| | | | | |
|---|---|---|---|---|
| NAV-WeedMD | 8.4 | g | 7.00% | 2.62% total CBD |
| CBD/THC oil 37-35 | | | | and 2.45% total |
| | | | | THC |
| Corn oil | 5.0 | g | 4.17% | |
| Peppermint oil | 0.12 | g | 0.10% | |
| Cremophor RH40 | 34 | g | 28.0% | |
| Vitamin E TPGS | 1.5 | g | 1.25% | |
| Propylene Glycol | 6.0 | g | 5.00% | |
| Ascorbic Acid | 0.3 | g | 2.00% | |
| Mixed Tocopherols | 1.5 | g | 1.25% | |
| Distilled water | 64 | g | 53.0% | |
| Subtotal | 120 | g | 100% | |

### Example 25

Emulsifier to CBD/THC Oil Ratio 6.23:1

| | | | | |
|---|---|---|---|---|
| NAV-WeedMD | 5.2 | g | 5.20% | 1.95% total CBD |
| CBD/THC oil 37-35 | | | | and 1.82% total |
| | | | | THC |
| Rapeseed oil | 3.0 | g | 3.00% | |
| MCT oil | 1.0 | g | 1.00% | |
| Vitamin E TPGS | 31.2 | g | 31.2% | |
| Polysorbate 80 | 1.2 | g | 1.20% | |
| Ascorbic Acid | 0.2 | g | 0.20% | |
| Mixed Tocopherols | 1.5 | g | 1.50% | |
| Distilled water | 56.7 | g | 56.7% | |
| Subtotal | 100 | g | 100% | |

MCT (medium-chain triglycerides) oil are triglycerides with two or three fatty acids having an aliphatic tail of 6-12 carbon atoms, i.e., medium-chain fatty acids (MCFAs). MCTs may include, for example, caproic acid, caprylic acid, capric acid and lauric acid, and may be straight chain fatty acids or branched chain fatty acids. MCT oils are commonly used as a diluent for lipid soluble bioactives, such as for cannabis tinctures.

### Example 26

Emulsifier to CBD/THC Oil Ratio 5.0:1

| | | | | |
|---|---|---|---|---|
| NAV-WeedMD | 6.8 | g | 6.8% | 2.55% total CBD |
| CBD/THC oil 37-35 | | | | and 2.38% total |
| | | | | THC |
| Canola oil | 3.0 | g | 3.0% | |
| Cremophor RH40 | 31.3 | g | 31% | |
| Polysorbate 80 | 1.2 | g | 1.2% | |
| Glycerol | 3.0 | g | 3.0% | |
| Ascorbic Acid | 0.2 | g | 0.2% | |
| Mixed Tocopherols | 1.5 | g | 1.5% | |
| Distilled water | 53.0 | g | 53% | |
| Subtotal | 100 | g | 100% | |

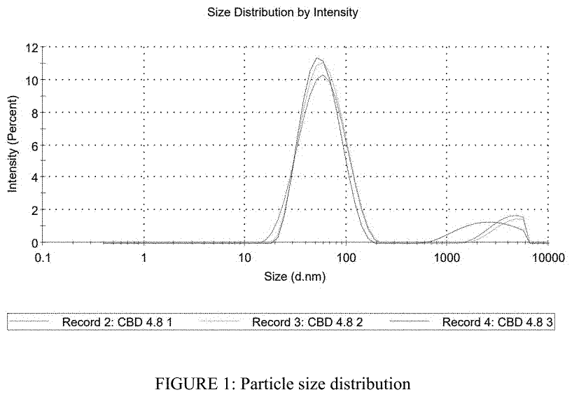

FIG. 1 is a representative graph of the results from a dynamic light scattering (DLS) experiment indicating the particle size distribution of a formulation of the present application, as obtained under the following conditions: Dispersant used: Water; Dispersant RI: 1.330; Viscosity (cP); 0.8872; Temperature (° C.): 25.0; Duration used (s): 50; Count Rate (kcps): 139.3; Measurement Position (mm): 0.85; Attenuator: 7. Results: Z-Average (d·nm): 58.05; Pdt: 0.367; Intercept: 0.559; Result Quality: Good.

| | Size (d.nm) | % Intensity | St Dev (d.n.) |
|---|---|---|---|
| Peak 1 | 63.60 | 89.8 | 30.47 |
| Peak 2 | 3821 | 10.2 | 1137 |
| Peak 3 | 0.000 | 0.0 | 0.000 |

The pre-drying emulsion (or emulsion) of the present application may include about 0.1% by weight to about 99% by weight additive or carrier, wherein the additive or carrier may also include a sweetener, a flavoring agent, a coloring agent, an anti-foaming agent, a nutrient, calcium or a calcium derivative, an energy-generating additive, an herbal supplement, a concentrated plant extract, a preservative, and/or combinations thereof.

In one aspect, the additive or carrier may include a gum and maltodextrin. In another aspect, the additive may be selected from the group consisting of crystalline cellulose, α-cellulose cross-linked carboxymethyl cellulose sodium, cross-linked starch, gelatin, casein, gum tragacanth, polyvinylpyrrolidone, chitin, chitosan, dextrin, kaolin, silicon dioxide hydrate, colloidal silicon dioxide, light silica, synthetic aluminum silicate, synthetic hydrotalcite, titanium oxide, dry aluminum hydroxy gel, magnesium carbonate, calcium carbonate, precipitated calcium carbonate, bentonite, aluminum magnesium metasilicate, calcium lactate, calcium stearate, calcium hydrogen phosphate, phosphoric acid anhydride, calcium hydrogen and talc. In one aspect, the additive comprises flowing agents selected from silicon dioxide and titanium oxide that promotes flowability or powdery characteristics of the dry powder. In one aspect, the emulsion comprises one or more additives selected from the group consisting of crystalline cellulose, α-cellulose, cross-linked carboxymethyl cellulose sodium, cross-linked starch, gelatin, casein, gum tragacanth, chitin, chitosan, calcium hydrogen phosphate, calcium hydrogen and precipitated calcium carbonate, and combinations thereof. In another aspect, the additive is comprised of wetting agents to assist in the dissolution of the dry powder, when the dry powder is dissolved in water. Such agents may include lecithin and the like.

In another aspect, the additives may include polymers that are added in an amount such that, where desired, the solution resulting from the re-dissolved powder of the present application remains substantially clear. The additive may include cellulosic polymers. Exemplary cellulosic polymers that may be used include hydroxypropyl methyl cellulose acetate, hydroxypropyl methyl cellulose, hydroxypropyl cellulose, methyl cellulose, hydroxyethyl methyl cellulose, hydroxyethyl cellulose acetate and hydroxyethyl ethyl cellulose. In another aspect, the polymers may include hydroxypropyl methyl cellulose and hydroxypropyl cellulose acetate. In another aspect, the polymers contain at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, alkoxyphthalic acid such as ethoxyphthalic acid and ethoxyisophthalic acid, and alkoxynicotinic acid such as ethoxynicotinic acid, etc.

In another aspect, exemplary cellulosic polymers may include hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, ethyl carboxymethyl cellulose, ethylbenzoic acid cellulose acetate and hydroxypropyl ethylbenzoic acid cellulose acetate. In another aspect, the cellulosic polymers may contain a non-aromatic carboxylate group, such as hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate and carboxymethyl ethyl cellulose.

In one embodiment, the composition further comprises an additive such as a sugar or sugar derivative, such as sucrose, glucose, lactose, levulose, fructose, maltose, ribose, dextrose, isomalt, sorbitol, mannitol, xylitol, lactitol, maltitol, pentatol, arabinose, pentose, xylose and galactose, and combinations thereof. The compositions of the present application may comprise from about 0.01 to 10% by weight, 10% to 25% by weight, or about 25% to 50% by weight of the above additive, relative to the weight of the dried powder formulation.

In one embodiment, the additives including coloring pigments, perfumes, flavoring and spices may be used in the appropriate concentration to obtain the desired color, flavors, aroma, taste and ultimate clarity of solution.

### Drying of Stabilized Surfactants and the CBD/THC Mixture:

One aspect of the drying method for the stabilized emulsion includes a spray drying method. The spray-drying method may include, for example, a method for spraying from a high-pressure nozzle. In another aspect, the method for spray-drying uses a centrifugal force, such as an atomizer. The gas or air that may be used for the spray drying includes heated air or hot air at a temperature sufficient to dry the powder having the desired moisture content. In one aspect, the gas is an inert gas such as nitrogen or nitrogen-enriched air.

In one aspect, the hot gas temperature may be at about 50° C. to 300° C., from 60° C. to 100° C., from 60° C. to 250° C., from 75° C. to 185° C., from 100° C. to 180° C., from 180° C. to 190° C., or about 180° C. The high pressure that may be used for the spray during process used in a high pressure nozzle may include about 10 to 1,000 psi, 100 to 800 psi or 200 to 500 psi. The spray drying may be carried out under conditions such that the residual water or residual moisture content of the dry powder may be controlled to about 1% to about 6%, 1% to 5%, 2% to 6%, 3% to 6% or about 3% to 5%.

In one aspect, the emulsions may then be sprayed dried in conventional spray drying equipment from commercial suppliers, such as Buchi, Niro, Yamato Chemical Co., Okawara Kakoki Co., and similar commercially available spray drier. Spray drying processes, such as rotary atomization, pressure atomization and two-fluid atomization may also be used. Examples of the devices used in these processes include Parubisu Mini-Spray GA-32 and Parubisu Spray Drier DL-41 (Yamato Chemical Co.) or Spray Drier CL-8, Spray Drier L-8, Spray Drier FL-12, Spray Drier FL-16 or Spray Drier FL-20, (Okawara Kakoki Co.), may be used for the spray drying method using rotary-disk atomizer. The nozzle of the atomizer that produces the powder of the present application may include, for example, nozzle types 1A, 1, 2A, 2, 3 (Yamato Chemical Co.) or similar commercially available nozzles, may be used for the above-mentioned spray drier. In addition, disks type MC-50, MC-65 or MC-85 (Okawara Kakoki Co.) may be used as rotary disks of the spray-drier atomizer.

In one aspect, the spray drying devices traditionally used for the industrial manufacture of a milk or coffee powder may also be employed in the present method. See Jensen J. D., Food Technology, June 60-71, 1975. In one aspect, the spray drying devices may include those described in U.S. Pat. No. 4,702,799. In one embodiment, operation of the spray drier may be performed at about 200-400° C. at the end of the spray nozzle where the rest of the device may be operated at a lower temperature which may reach the air outlet temperature, such as the sprayer described in U.S. Pat. No. 3,065,076. In another aspect, the spray-drying apparatus used in the process of the present application may be any of the various commercially available apparati. Representative examples of spray drying apparati are the Anhydro Dryers (Anhydro Corp., Attleboro Falls, Mass.), the Niro Dryer (Niro Atomizer Ltd., Copenhagen, Denmark) or a Leaflash apparatus (CCM Sulzer). In one aspect, a spray-drier with a pressure nozzle may be used.

In another aspect, the powder obtained from the drying process may comprise 10% by weight, 20% by weight, 30% by weight, 40% by weight, 50% by weight, 60% by weight, 70% by weight, 80% by weight, or 90% by weight or more of particles having an average particle size in the range from about 5 to 1,000 microns, from about 10 to 500 microns, from 10 to 350 microns, from 20 to 250 microns, from 40 to 200 microns, or about 50 to 150 microns. In one aspect, the powder obtained from the drying process comprises of about 20% to 80% by weight of particles with an average particle size of 50 to 150 microns.

The dry composition of the present application may be formulated to provide a dry powder that is stable, and may form a partially turbid solution, a milky or cloudy solution, or a clear solution as desired. Where a substantially clear solution or composition is not desired, such as a milky or cloudy solution or composition is desired as obtained from the dry powder, the ratio of the solubilizing agent, such as TPGS or TPGS-750-M, to the CBD/THC mixture may be reduced. For example, the ratio (w/w or wt/wt) of the emulsion, such as TPGS or TPGS-750-M, to CBD/THC mixture may be reduced to a range of about 2:1 to about 1.5:1, 1.3:1, 1:1, or about 0.9:1 or less.

The dry powder formulation of the present application provides CBD/THC mixture that are stable to decomposition. Without being bound by any theory presented herein, it is believed that the judicious selection of the solid support allows the encapsulation of the CBD/THC mixture, provides substantially no surface oil and shields the CBD/THC mixture from oxidation by exposure to ambient air. In addition, the dry powder formulation is readily re-dissolved in water and forms a clear solution.

The concentrated powder may be prepared as dry preparations, such as, for example, a powder, a granular material, a crystalline material, other types of dry particle preparations or combinations thereof. In one aspect, the dry preparations may be prepared by mixing the ingredients and compositions, as disclosed herein, to form a concentrated solution, and then drying the solution to a dry powder form by conventional drying methods. Representative drying methods may include for example, lyophilization (or freeze drying), spray drying, fluid bed drying, drum drying, pulse combustion drying and various combinations thereof. In one aspect, the method is a spray drying method.

### Surfactants or Solubilizing Agents:

One or more surfactants (or solubilizing agents), or a mixture of surfactants may be used in the present formulations. Representative surfactants employed may include: HLB≥10 surfactants such as Poloxamer 188, Polysorbate 80, Polysorbate 20, Vit E-TPGS, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); HLB 8-12 such as Polysorbate 85, polyglyceryl-6-dioleate (Caprol MPGO), Mixtures of high and low HLB emulsifiers; and LB≤8 such as Sorbitan monooleate (Span 80), Capmul MCM, Maisine 35-1, glyceryl monooleate, glyceryl monolinoleate, PEG-6-glyceryl oleate (Labrafil M 1944 CS), PEG-6-glyceryl linoleate (Labrafil M 2125 CS), oleic acid, linoleic acid, propylene glycol monocaprylate (e.g. Capmul PG-8 or Capryol 90), propylene glycol monolaurate (e.g., Capmul PG-12 or Lauroglycol 90), polyglyceryl-3 dioleate (Plurol Oleique CC497), polyglyceryl-3 diisostearate (Plurol Diisostearique) and lecithin with and without bile salts. NAV-WeedMD CBD/THC oil 49-38:

A mixture of THC/CBD oil (7.70 g, THC/CBD oil from NAV-WeedMD extract contains 49.2% CBD and 38.4% THC and some dark-colored impurities) is added to a 250 mL high pressure Corning glass bottle with screw-on cap and the THC/CBD oil in the glass bottle was warmed with a hot water bath (heated via a hot plate) to 60-70° C. for 3 minutes.

Cremophor RH40 (50 g) was added to a separate 100 mL beaker, and the Cremophor RH40 was heated in a microwave (1000 Watts of power) at high power for about 90 seconds until the Cremophor RH40 was free flowing. 33.1 g (32.1 mL, density 1.03 g/mL at 60° C.) of the heated Cremophor RH40 was added to the 250 mL bottle containing the THC/CBD oil. 4.70 g of canola oil, 1.5 g of mixed tocopherol, 0.2 g of ascorbic acid, 1.7 g of Vitamin E TPGS (tocoferosolan), 5.0 g of glycerol, and 56.2 g of distilled water was added to the 250 mL Corning glass bottle. The screw cap was screwed onto the Corning glass bottle and tightened. The glass bottle was placed into a microwave oven and heated for 3 consecutive heating and depressurization periods at high power as follows: Heating at 55 seconds followed by opening of the cap to release the pressure; heating again for 30 seconds followed by opening of the cap to release the pressure, and heating again for 20 seconds followed by opening the cap to release the pressure build-up, and then re-tighten up the cap to seal the bottle.

The bottle is then immediately placed in under running cold water (about room temperature) with moderate swirling for at least 30 seconds, and the cooling was performed at no more than about 45 seconds. After the cooling, the solution became clear and transparent, with a dark amber in color. Due to the dark color, it is not obvious whether the solution is clear and homogeneous, so the solution is diluted for visualization, as follows. 10 mL of the dark solution is then diluted with 90 mL of water to form a 100 mL diluted solution. The diluted solution shows that the THC/CBD solution is clear and homogeneous.

In cases where the solution appears opaque, cloudy or inhomogeneous, the 3-steps microwave heating at 55 seconds, 30 seconds and 20 seconds is repeated, followed by the same water cooling step is performed again to provide a clear, homogenous solution.

To the homogeneous solution is added ascorbic acid (0.3 g), sodium benzoate (0.060 g), and potassium sorbate (0.12 g), where the ascorbic acid, sodium benzoate and potassium sorbate dissolve in the homogeneous solution. The homogeneous solution is filtered through a SCILOGEX 3 μm Spare Hydrophobic Filter into a 200 mL storage container and sealed with a screw cap. The storage container is stored away from direct sunlight at room temperature.

As described herein, the methods may be used for batch processing to prepare the composition. However, continuous processing of the described methods may also be employed, using 2 feeds of liquids, a water feed containing the water soluble components of the composition, and an oil feed containing the fat soluble components of the composition. The 2 feeds are dosed in their desired proportions using suitably designed pumps (volumetric or positive displacement pumps) into small mixing tank, or into a static mixer. The combined feeds are then passed over 2 suitably designed sequentially mounted heat exchangers [HEX](i.e. plate HEX, shell and tube HEX), the first one heating the combined mixed feed to the desired engineered target temperature, the second one cooling the hot mixed feed down to below 50° C. for collection.

Emulsions prepared according to the above procedure may be dried using various drying methods as provided herein. In one embodiment, the emulsions may be dried using the spray drying methods as described herein. The spray dried composition comprises water content from about 1% to about 10%, 1% to 6%, 2% to 5%, 3% to 4%, 1% to 3%, 2% to 3%, 3% to 6%, 3% to 5%, or about 3% to 4%. Accordingly, the clarity or homogeneity of the aqueous solution containing the compositions as described herein may be controlled by the amount residual water remaining in the dried powders.

The compositions and methods of the present application are illustrated by the examples described herein. These examples are offered to illustrate, but not to limit the claimed present application.

Solubility, clarity and stability results of the solution prepared according to the procedures as described herein demonstrate that the formulations as described herein maintains clarity and stability for the desired period of time under the storage conditions. Bioavailability of Emulsions:

In order to determine the extent of the relative bioavailability increase of a representative composition described herein (experimental product) over a standard commercial oily solution (control product), using the same balanced source of a decarboxylated natural extract concentrate of CBD/THC, a single center, randomized, double blind, placebo-controlled, parallel bioavailability study was conducted with 32 subjects enrolled (16 per arm). Dosage was standardized to 10 mg of THC. Primary outcomes were Area under the curve (AUC_{0-48 h}) (overall absorption), maximum concentration (C_{max, 0-48 h}) (bioavailability), and time to maximum concentration (T_{max, 0-48 h}) (speed of absorption) for CBD, Δ⁹-THC, 11-OH-THC, 11-NOR-9-Carboxy-Δ⁹-THC (11-NOR-9-Carboxy-THC) and CBDA. Secondary outcome was a subjective evaluation of drug effects assessed using modified Drug Effects Questionnaire (DEQ-5) for the control product and the experimental product. Relevant experimental descriptions of several study aspects are detailed below.

Administration: A pre-dose plasma (via IV catheter) and a urine sample is collected from the participant before the administration of the emulsions/formulations. A selected emulsion formulation and comparator products are prepared as follows: (372 mg calculated according to Example 22) of the experimental product and control product, in an amount to correspond to a 10 mg target delivery of THC, is each mixed with 7 gm of plain oats (28 gm package divided into four portions to give 7 gm) prepared in water. The bowl in which the product is mixed is rinsed with 100 ml water and the participant consumes the complete amount. After the product mixture is consumed, the participant is provided with an 8 oz glass of water that is then consumed in its entirety within 1-2 minutes.

Blood sampling: After a selected period of time including T=15 minutes, 30 minutes, 45 minutes, 1 hr, 1.5 hrs, 2 hrs, 2.5 hrs, 3 hrs, 4 hrs, 5 hrs, 6 hrs, 8 hrs and 12 hrs, samples of blood is taken for the analysis of CBD, Δ⁹-THC and their metabolites in plasma. The samples are also analyzed for HbAlc, CBC, electrolytes (Na, K, Cl, Ca), creatinine, estimated glomerular filtration rate (eGFR), aspartate aminotransferase (AST), alanine aminotransferase (ALT), and bilirubin. Urine samples are also obtained at the following time ranges: 1-4 hrs, 4-8 hrs and 8-12 hrs.

### Blood Sample Collection

IV blood samples are be collected from participants using standard protocol.

### Laboratory Analysis:

Blood samples are drawn from the participants at screening (Visit 1) and baseline (Visit 2, Day 1) as indicated in the Schedule of Assessments. Blood draws are performed from the participant's arm via IV catheter as noted. Protection of subject confidentiality will extend to all data generated from the assaying of these samples that are alphanumerically coded.

At screening (Visit 1), 13 mL of whole blood is collected in:
1. Two 4 mL EDTA vacutainer tubes to generate plasma for:
   a. CBC analysis (one tube)
   b. Hb1Ac analysis (one tube)
2. one 5 mL SST vacutainer tube to generate serum for:
   a. Chemistry analysis (one tube)
      At baseline (Visit 2), 61 mL of whole blood is collected in:
1. 14 4 mL EDTA vacutainer tubes to generate plasma for:
   a. CBD, Δ⁹-THC, 11-OH-THC, 11-NOR-9-CARBOXY-Δ⁹-THC and CBD acid analysis (one tube per time point)
2. 1 5 mL SST vacutainer tube to generate serum for:
   a. Chemistry analysis (one tube)

The total blood volume collection for the laboratory assessments listed above is about 75 mL, over the period from screening to end of study (about 45 days). At the study visit, blood loss per volunteer is not expected to exceed 61 mL. Additional blood samples may be collected during the course of the study in order to perform or repeat laboratory tests outlined in the Schedule of Assessments. LifeLabs, a central laboratory is used to measure blood parameters.

### Sample Processing for CBD and Δ⁹-THC Analysis:

Plasma and urine samples are collected at KGK Science. Analysis for CBDΔ⁹-THC, 11-OH-THC, 11-NOR-9-CARBOXY-Δ⁹-THC and CBD acid in plasma is performed by Altasciences by LCMS and is validated over the analytical range of 0.5-150 ng/mL of CBD and 5-1500 ng/mL of Δ⁹-THC.

### Statistical Analysis:

Numerical endpoints are formally compared between products by an analysis of variance (ANOVA) and/or analysis of covariance (ANCOVA), where the value of the variable at the end of the supplementation period or the change from baseline to the end of the study is the dependent variable, the supplement group or Placebo is the factor of interest, and the value of the variable at baseline (pre-supplementation) is the covariate. Categorical endpoints will be compared between products by the Fisher Exact Test.

### Schedule of Assessments: (N=32)

| | | Visit 2 | |
|---|---|---|---|
| | Visit 1 | Baseline | Visit 3 |
| | Screening | Day 1 | Day 15 |
| Informed consent | X | | |
| Review inclusion/exclusion criteria | X | X | |
| Review medical history | X | | |
| Review concomitant therapies | X | X | |
| Height*, weight, heart rate, blood pressure and BMI | X | X | |
| * only measured at visit 1 | | | |
| Urine pregnancy test | X | X | |
| Physical examination | | X | |
| Randomization | | X | |
| Laboratory tests: | X | X | |
| blood sample collection | | | |
| CBC, electrolytes, HbA1c*, creatinine, AST, ALT, | | | |
| bilirubin and estimated glomerular filtration rate | | | |
| (eGFR) | | | |
| *only measured at Visit 1 | | | |
| Blood Draw: | | X | |
| CBD, Δ⁹-THC, 11-OH-THC, 11-NOR-9- | | | |
| CARBOXY-Δ9-THC and CBD acid analysis at | | | |
| time = 0 minutes (pre-dose), 15 minutes, 30 minutes, | | | |
| 45 minutes, 1 hr, 1.5 hrs, 2 hours, 2.5 hrs, 3 hrs, 4 | | | |
| hrs, 5 hrs, 6 hrs, 8 hrs, and 12 hrs post dose | | | |
| Urine Collection for CBD and Δ⁹-THC analysis at | | X | |
| Time = 0 minutes, 1-4 hrs, 4-8 hrs and 8-12 hrs | | | |
| Modified Drug Effects Questionnaire-5: | | X | X |
| At time = 0 minutes (pre-dose), 15 minutes, 30 | | | |
| minutes, 45 minutes, 1 hr, 2 hrs, 3 hrs, 4 hrs, 6 hrs, 8 | | | |
| hrs, 10 hrs, 12 hrs | | | |
| Food diary dispensed | X | | |
| Food diary returned | | X | |
| IP dispensed and consumed | | X | X |
| Meals dispensed and consumed (Breakfast, lunch and | | X | X |
| dinner) | | | |
| AE diary dispensed | X | | |
| AE diary returned | | X | |
| Compliance recorded | | X | X |

As disclosed herein, the measured bioavailability of THC, 11-OH-THC and CBD, based on the administration of THC and CBD, are determined or measured independently. Using various formulations comprising >10 mg THC and >12.6 mg CBD, for example, the measured ranges of values for THC, 11-OH-THC and CBD are:

### For THC:

The AUC may be determined at times=0 h, 5 h, 10 h, 15 h, 20 h, 24 h, 35 h, 40 h, 45 h and 48 h, to provide 11 h*ng/mL, 15 h*ng/mL, 20 h*ng/mL, 30 h*ng/mL, 40 h*ng/mL, 50 h*ng/mL, 60 h*ng/mL, 70 h*ng/mL, 80 h*ng/mL, 90 h*ng/mL, 100 h*ng/mL and 110 h*ng/mL; with a Cmax of 3.6 ng/mL, 5 ng/mL, 10 ng/mL, 15 ng/mL, 20 ng/mL, 25 ng/mL, 30 ng/mL, 35 ng/mL and 36 ng/mL; at a Tmax of 10 mins, 20 mins, 30 mins, 40 mins, 50 mins, 60 mins, 70 mins, 80 mins and 90 mins.

### For 11-OH-THC:

The AUC may be determined at times=0 h, 5 h, 10 h, 15 h, 20 h, 24 h, 35 h, 40 h, 45 h and 48 h, to provide 26.00 h*ng/mL, 35 h*ng/mL, 50 h*ng/mL, 75 h*ng/mL, 100 h*ng/mL, 100 h*ng/mL, 125 h*ng/mL, 150 h*ng/mL, 175 h*ng/mL, 200 h*ng/mL, 225 h*ng/mL and 260 h*ng/mL; with a Cmax of 4.2 ng/mL, 7 ng/mL, 10 ng/mL, 15 ng/mL, 20 ng/mL, 25 ng/mL, 30 ng/mL, 35 ng/mL, 40 ng/mL and 42 ng/mL; at a Tmax of 10 mins, 20 mins, 30 mins, 40 mins, 50 mins, 60 mins, 70 mins, 80 mins, 90 mins, 115 mins, 120 mins, 130 mins, 135 mins, 140 mins, 145 mins.

### For CBD:

The AUC may be determined at times=0 h, 5 h, 10 h, 15 h, 20 h, 24 h, 35 h, 40 h, 45 h and 48 h, to provide 5 h*ng/mL, 6 h*ng/mL, 8 h*ng/mL, 11 h*ng/mL, 15 h*ng/mL, 20 h*ng/mL, 30 h*ng/mL, 40 h*ng/mL, 50 h*ng/mL and 60 h*ng/mL; with a Cmax of 1.4 ng/mL, 2 ng/mL, 3 ng/mL, 4.2 ng/mL, 7 ng/mL, 10 ng/mL, 14 ng/mL; at a Tmax of 10 mins, 20 mins, 30 mins, 40 mins, 50 mins, 60 mins, 70 mins, 80 mins and 85 mins.

### Therapeutic Applications:

Therapeutical methods using the emulsion and formulations of the present application may be used effectively to treat, reduce or mitigate a variety of diseases selected from the group consisting of neurological disorders and neurological diseases such as neuropathic pain, chronic pain, migraine, Post Traumatic Stress Disorder (PTSD), pruritus, rheumatoid arthritis, sleep apnea, Huntington disease 1,3-butanediol e (HD), Alzheimer's disease, Parkinson's disease, amyotrophic lateral sclerosis (ALS), hypertension, incontinence, diabetes, hepatitis C, multiple sclerosis (MS), rheumatoid arthritis (RA), osteoporosis, dystonia, epilepsy, fibromyalgia and Tourette syndrome (TS), gastrointestinal disorders including functional bowel diseases such as irritable bowel syndrome (IBS) and inflammatory bowel diseases such as Crohn's disease (CD) and colitis, hepatitis C and HIV infections. In another variation, there is provided a method for the treatment of nausea and vomiting associated with chemotherapy and appetite stimulation of AIDS patients suffering from the wasting syndrome, the method comprises the administration of the stabilized, aqueous and purified cannabis oil emulsion as recited herein.

Accordingly, there is provided a stabilized, aqueous and purified cannabis oil emulsion comprising: a) CBD and THC (CBD/THC) wherein the ratio of CBD:THC by wt/wt is from 1,050:1 to 1:1,050, and b) at least one emulsifier selected from the group consisting of Poloxamer 188, Polysorbate 80, Polysorbate 20, Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; wherein the emulsion is stable for a period of at least 30 days when stored at about 20-30° C.

In another aspect of the above cannabis oil emulsion, the administration of the cannabis oil emulsion comprising of no less than 10 mg of CBD and no less than 10 mg of THC to a subject provides a bioavailability of CBD, 11-OH-THC, 11-NOR-9-Carboxy-THC and THC in plasma after the administration resulting in: 1) a mean area under the curve (AUC_{0-48 h}) of at least a 12 h*ng/mL for THC, at least 28 h*ng/mL for 11-OH-THC, at least 200 h*ng/mL for 11-NOR-9-Carboxy-THC, or at least 5 h*ng/mL for CBD; 2) a mean maximum peak concentration (C_{max, 0-48 h}) of at least 3.9 ng/mL THC, at least 4.6 ng/mL 11-OH-THC, at least 50 ng/mL for 11-NOR-9-Carboxy-THC, or at least 1.2 ng/mL CBD; or 3) a mean time to maximum peak concentration (T_{max, 0-48 h}) of at most 1.5 h for THC, at most 2 h for 11-OH-THC, at most 2 h for 11-NOR-9-Carboxy-THC, or at most 1.5 h for CBD. As used herein, the clause “no less than” may be used interchangeably with the clause “at least or equal to”.

In one aspect of the above cannabis oil emulsion, the administration of no less than 10 mg of CBD and no less than 10 mg of THC; or >12.6 mg of CBD and >10.0 mg of THC; to a subject provides a bioavailability of CBD, 11-OH-THC and THC in plasma after the administration resulting in: 1) an average area under the curve (AUC_{0-48 h}) of at least a 11 h*ng/mL for THC, at least 26 h*ng/mL for 11-OH-THC, and at least 6 h*ng/mL for CBD; 2) an average maximum peak concentration (C_{max, 0-48 h}) of at least a 3.6 ng/mL THC, at least 4.2 ng/mL 11-OH-THC, and at least 1.4 ng/mL CBD; or 3) an average time to maximum peak concentration (T_{max, 0-48 h}) of at most 40 min for THC, at most 80 min for 11-OH-THC, and at most 40 min for CBD. As used herein, the clause “at most” may be used interchangeably with the clause “less than or equal to”. In one variation of the above, the Tmax for THC, 11-OH-THC and CBD was about 10 min (minutes), 15 min, 20 min, 35 min, 30 min, 35 min, 45 min, 50 min or about 1 h.

In one variation, the emulsion comprising, for example, a 20 mg/ml of CBD or THC provides a maximal plasma levels after the administration by oral dosing of the emulsion, are greater than about 15 ng/ml, 20 ng/ml, 25 ng/ml, 30 ng/ml, 35 ng/ml, 40 ng/ml, or more than 50 ng/ml. In another variation, the emulsion comprising a 20 mg/ml of CBD or THC provides a pharmacokinetic profile of Cmax of at least about 15 ng/ml, 20 ng/ml, 25 ng/ml, 30 ng/ml, 35 ng/ml, 40 ng/ml, or more than 50 ng/ml; an AUC (0-12) of at least about 10 h*ng/mL (hours times nanogram per milliliter), 15 h*ng/mL, 20 h*ng/mL, 25 h*ng/mL, 30 h*ng/mL, 35 h*ng/mL, 40 h*ng/mL or at least about 50 h*ng/mL; or a Tmax of 0.25 h, 0.5 h, 1 h, 1.5 h, 2 h, 3 h, 4 h or 5 h. In one variation of the above pharmacokinetic profile, the Tmax for CBD and/or THC was about 10 min (minutes), 15 min, 20 min, 35 min, 30 min, 35 min, 45 min, 50 min or about 1 h.

In another variation, at least about 30%, 40%, 45%, 50%, 55% or 60%, 70% or about 80% of the administered dose of CBD or THC reaches systemic circulation with the above maximal plasma levels after administration. It is noted that these measured maximal plasma levels, Cmax profiles and AUC, for example, measure the specific levels of CBD and THC independently, although a combination or a mixture of both CBD and THC may be administered and then measured as noted above.

In one variation, the mixture of the emulsifiers is a mixture of 2 different emulsifiers, or a mixture of 3 different emulsifiers. In one variation, the THC is selected from the group consisting of Δ⁹-THC, 11-OH-THC and 11-nor-9-carboxy-Δ⁹-THC. In another variation, the bioavailability of the CBD/THC measured in plasma is at least 20%, 30%, 40%, 50%, 100%, 150%, 200%, 250%, 300%, 350%, 400% or more than 500% greater than CBD, THC or the above mixture of CBD and THC when administered in the absence of the above cited cannabis oil emulsion of the present application comprising the at least one emulsifier. In one variation, the ratio of CBD:THC by wt/wt is from 1,050:1 to 1:1,050.

In another aspect of the above cited cannabis oil emulsion, wherein the administration of no less than 10 mg of CBD and no less than 10 mg of THC to a subject provides relative pharmacokinetic values for CBD, 11-OH-THC, 11-NOR-9-Carboxy-THC, and/or THC in plasma after the administration, when compared to the administration to a subject of a cannabis oil composition comprising CBD and THC dissolved in MCT oil (the Comparator formulation) comprising no less than 10 mg of CBD and no less than 10 mg of THC, resulting in: 1) a relative mean area under the curve (AUC_{0-48 h}) increase of the above cannabis oil emulsion over the Comparator formulation of at least 25% for THC, at least 25% for 11-OH-THC, at least 25% for 11-NOR-9-Carboxy-THC, or at least 25% for CBD; 2) a relative mean maximum peak concentration (C_{max, 0-48 h}) increase of the above cannabis oil emulsion over the Comparator formulation of at least 25% for THC, at least 25% for 11-OH-THC, at least 25% for 11-NOR-9-Carboxy-THC, or at least 25% for CBD; or 3) a relative mean time to maximum peak concentration (T_{max, 0-48 h}) reduction of the above cited cannabis oil emulsion over the Comparator formulation of at least 25% for THC, at least 25% for 11-OH-THC, at least 25% for 11-NOR-9-Carboxy-THC, or at least 25% for CBD.

In one variation of the above relative pharmacokinetic values, the administration results in: 1) a relative mean area under the curve (AUC_{0-48 h}) increase of the above cannabis oil emulsion over the Comparator formulation of at least 10%, 25%, 30%, 50%, 75%, 100%, 125%, 150%, 175%, 200%, 300%, 400%, 500%, 600%, 700%, 800%, 900% or more than 1,000% for THC, at least 25%, 25%, 30%, 50%, 75%, 100%, 125%, 150%, 175%, 200%, 300%, 400%, 500%, 600%, 700%, 800%, 900% or more than 1,000% for 11-OH-THC, at least 10%, 25%, 30%, 50%, 75%, 100%, 125%, 150%, 175%, 200%, 300%, 400%, 500%, 600%, 700%, 800%, 900% or more than 1,000% for 11-NOR-9-Carboxy-THC, or at least 10%, 25%, 30%, 50%, 75%, 100%, 125%, 150%, 175%, 200%, 300%, 400%, 500%, 600%, 700%, 800%, 900% or more than 1,000% for CBD; 2) a relative mean maximum peak concentration (C_{max, 0-48 h}) increase of the above cannabis oil emulsion over the Comparator formulation of at least 10%, 25%, 30%, 50%, 75%, 100%, 125%, 150%, 175%, 200%, 300%, 400%, 500%, 600%, 700%, 800%, 900% or more than 1,000% for THC, at least 10%, 25%, 30%, 50%, 75%, 100%, 125%, 150%, 175%, 200%, 300%, 400%, 500%, 600%, 700%, 800%, 900% or more than 1,000% for 11-OH-THC, at least 10%, 25%, 30%, 50%, 75%, 100%, 125%, 150%, 175%, 200%, 300%, 400%, 500%, 600%, 700%, 800%, 900% or more than 1,000% for 11-NOR-9-Carboxy-THC, or at least 10%, 25%, 30%, 50%, 75%, 100%, 125%, 150%, 175%, 200%, 300%, 400%, 500%, 600%, 700%, 800%, 900% or more than 1,000% for CBD; or 3) a relative mean time to maximum peak concentration (T_{max, 0-48 h}) reduction of the above cannabis oil emulsion over the Comparator formulation of at least 10%, 25%, 30%, 50%, 75% or 95% for THC, at least 10%, 25%, 30%, 50%, 75%, 85% or 95% for 11-OH-THC, at least 10%, 25%, 30%, 50%, 75%, 85% or 95% for 11-NOR-9-Carboxy-THC, or at least 10%, 25%, 30%, 50%, 75%, 85% or 95% for CBD.

In another aspect of the cannabis oil emulsion, the emulsifier is GRAS or a food grade emulsifier. In another aspect of the cannabis oil emulsion, the ratio of CBD:THC is 1:1. In another aspect, the cannabis oil emulsion further comprises one or more co-solvents selected from the group consisting of ethanol, glycerol, propylene glycol, 1,3-propanediol, butylene glycol, erythritol, xylitol, mannitol, sorbitol, isomalt, polyethylene glycols (e.g., PEG-400), and a combination thereof. In another aspect, the cannabis oil emulsion further comprises one or more vegetable oil selected from the group consisting of arachis oil, olive oil, sesame oil or coconut oil and a mineral oil, or combinations thereof. In another aspect, the cannabis oil emulsion further comprises one or more oils selected from the group consisting of cannabis oil (hemp oil), coconut oil, cottonseed oil, soybean oil, sunflower oil, castor oil, corn oil, olive oil, palm oil, peanut oil, almond oil, sesame oil, rapeseed oil, peppermint oil, canola oil, palm kernel oil, hydrogenated soybean oil, medium-chain triglycerides (MCLs), short-chain triglycerides, glyceryl esters of saturated fatty acids, glyceryl behenate, glyceryl distearate, glyceryl isostearate, glyceryl aurate, glyceryl monooleate, glyceryl monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and combinations thereof. In another aspect, the cannabis oil emulsion further comprises one or more masking or flavoring component selected from the group consisting of natural cinnamon oil, peppermint oil, clove oil, bay oil, thyme oil, artificial, natural or synthetic fruit flavors selected from the group consisting of vanilla, chocolate, coffee, cocoa, and citrus oil selected from the group consisting of lemon, lime, orange, grape, grapefruit, and fruit essences selected from the group consisting of apple, pear, peach, strawberry, watermelon, raspberry, cherry, plum, pineapple and apricot, or combinations thereof.

In yet another aspect, the cannabis oil emulsion further comprises one or more additives comprising: a) a stabilizer or antioxidant selected from the group consisting of tocopherols, flavonoids, catechins, superoxide dismutase, lecithin, gamma oryzanol, vitamins A, C (ascorbic acid) and E including homologues and isomers thereof, camosol, carnosic acid and rosmanol, hawthorn extract and proanthocyanidins, or combinations thereof; and b) a reducing agent selected from the group consisting of L-ascorbic acid-6-palmitate, vitamin C and ubiquinol, or mixtures thereof. In another aspect, the cannabis oil emulsion further comprises a metal chelator selected from the group consisting of ethylenediaminetetraacetic acid (EDTA), disodium EDTA and calcium disodium EDTA and mixtures thereof. In another aspect, the cannabis oil emulsion further comprises an absorption enhancer or bioavailability enhancer selected from the group consisting of medium chain fatty acids, omega-3 fatty acids, capric acid, caprylic acid, (8-[2-hydroxybenzoyl]-amino)caprylic acid, N-(10-[2-hydroxybenzoyl]-amino)decanoic acid, N-(8-[2-hydroxybenzoyl]-amino)caprylic acid (SNAC, salcaprozate sodium), 8-(N-2-hydroxy-5-chloro-benzoyl)-amino-caprylic acid (5-CNAC), N-(10-[2-hydroxybenzoyl]-amino)decanoic acid, alkylglycosides, chitosan, trimethylated chitosan, protease inhibitors, β-glycoprotein inhibitors, dodecyl-2-N,N-dimethylamino propionate (DDAIP), calcium chelating agents (i.e. ethylene glycol tetraacetic acid, ethylene diamine tetraacetic acid (EDTA), salicylic acid, flavonoids (i.e. quercetin ((2-(3,4-dihydroxyphenyl)-3,5,7-trihydroxy-4Hchromen-4-one), luteolin), isoflavones (i.e. genistein (5,7-dihydroxy-3-(4-hydroxyphenyl)chromen-4-one)), flavonoid glycosides (naringin), alkaloids (i.e. sinomenine (7,8-didehydro-4-hydroxy-3,7-dimethoxy-17-methylmorphinan-6-one), triterpenoid saponins (glycyrrhizin [(3,18)-30-hydroxy-11,30-dioxoolean-12-en-3-yl 2-O-glucopyranuronosyl-Dglucopyranosiduronic acid]), nitrile glycosides, phytomolecules (i.e. lysergol, allicin (garlic)), terpenes (ginkgolide A, B, C and J), ginsenosides, epigallocatechin, epigallocatechin gallate, phenanthrene, cuminumcyminum Linn, herb, ginger, aloe vera, capsaicin, colchicine, vincristine, matrine, ammonium glycyrrhizinate, beeswax, piperine, trikatu, and their pharmaceutically acceptable salts (i.e. sodium), or derivatives (i.e. esters). In another aspect of the cannabis oil emulsion, the range of the ratio of the emulsifier to CBD/THC is between 11.0:1.0 to 1.0:1.0, 7.0:1.0 to 1.5:1.0 or 5.0:1.0 to 2.0:1.0.

In another aspect of the cannabis oil emulsion, the CBD/THC concentration in the emulsion is about 10%, 9%, 8%, 7%, 5%, 3%, 2%, 1%, 0.5%, 0.1% or 0.01% or less. In another aspect of the cannabis oil emulsion, the emulsion comprises of particle size is less than about 500 nm, less than 300 nm, less than 200 nm, less than 100 nm, less than 80 nm, less than 60 nm; less than 40 nm; or between about 20 and 30 nm, as measured by DLS. In another aspect of the cannabis oil emulsion, the emulsion has a measured Nephelometric Turbidities in a range of about 10 to 1000, 20 to 300 or 30 to 100.

In another embodiment, there is provided a method for the preparation of the cannabis oil emulsion of the above, wherein the method comprises: a) weighing the components of the above emulsion into a reaction container; b) heating the combined emulsion to a temperature from about 25° C. to about 130° C. with agitation for a sufficient amount of time to prepare the emulsion; and c) cooling the emulsion to about 25° C. In one aspect of the above method, the preparation is performed under nitrogen atmosphere. In another aspect of the method, the heating of the cannabis oil emulsion is performed to a temperature of about 70° C. to 100° C., or about 80° C. to 95° C. In another aspect of the above method, the cooling of the cannabis oil emulsion is performed using an external ice bath or the equivalent. In another embodiment of the cannabis oil emulsion prepared by the above method, the resulting stable emulsion has a shelf stability of at least 3 months, 6 months or 12 months when stored at about 0° C. to 50° C., or about 25° C. to 35° C. In another aspect of the cannabis oil emulsion, the natural odor of the CBD/THC emulsion is effectively masked and provides a pleasant taste for oral consumption. In another aspect of the cannabis oil emulsion, the oxidative stability of the emulsion is enhanced over that of a CBD/THC mixture by at least 3 months when the CBD/THC mixture is stored at about 0° C. to 50° C.

In another embodiment, there is provided a liquid nutritional composition selected from the group consisting of beverages, soft drinks, carbonated beverages, enhanced waters, gels, gelatins, concentrates, beverage enhancers, wherein the composition is prepared by the method comprising: a) obtaining the cannabis oil emulsion as described above; and b) diluting the cannabis oil emulsion to a desired liquid nutritional composition.

In another embodiment, there is provided a method for treating or alleviating a neurological disorders and neurological diseases selected from neuropathic pain, chronic pain, migraine, Post Traumatic Stress Disorder (PTSD), Huntington disease (HD), Alzheimer's disease, Parkinson's disease, amyotrophic lateral sclerosis (ALS), multiple sclerosis (MS), epilepsy and dystonia to a subject in need of such treatment, the method comprising the administration to the subject in need thereof a therapeutically effective amount of the stabilized, aqueous and purified cannabis oil emulsion of any of the above recited embodiments and aspects. In another embodiment, there is provided a method for treating or alleviating a disease or disorder selected from the group consisting of pruritus, rheumatoid arthritis, sleep apnea, hypertension, incontinence, diabetes, hepatitis C, rheumatoid arthritis (RA), osteoporosis, fibromyalgia and Tourette syndrome (TS), gastrointestinal disorders including functional bowel diseases such as irritable bowel syndrome (IBS) and inflammatory bowel diseases such as Crohn's disease (CD) and colitis, hepatitis C and HIV infections to a subject in need of such treatment, the method comprising the administration to the subject in need thereof a therapeutically effective amount of the stabilized, aqueous and purified cannabis oil emulsion of any of the above recited embodiments and aspects. In yet another embodiment, there is provided a method for treating or alleviating a disease or disorder selected from the group consisting of nausea and vomiting associated with chemotherapy and appetite stimulation of AIDS patients suffering from the wasting syndrome, the method comprises the administration to the patient in need thereof the stabilized, aqueous and purified cannabis oil emulsion of any one of the above embodiments and aspects.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

What is claimed is:

1. A stabilized, aqueous and purified cannabis oil emulsion comprising:
a) CBD and THC (CBD/THC) wherein the ratio of CBD:THC by wt/wt is from 1,050:1 to 1:1,050, and
b) at least one emulsifier selected from the group consisting of Poloxamer 188, Polysorbate 80, Polysorbate 20, Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof;
wherein the emulsion is stable for a period of at least 30 days when stored at about 20-30° C.

2. The cannabis oil emulsion of claim 1, wherein an administration of the cannabis oil emulsion comprising of no less than 10 mg of CBD and no less than 10 mg of THC to a subject provides a bioavailability of CBD, 11-OH-THC, 11-NOR-9-Carboxy-THC and THC in plasma after the administration resulting in:
1) a mean area under the curve (AUC_{0-48 h}) of at least a 12 h*ng/mL for THC, at least 28 h*ng/mL for 11-OH-THC, at least 200 h*ng/mL for 11-NOR-9-Carboxy-THC, or at least 5 h*ng/mL for CBD;
2) a mean maximum peak concentration (C_{max, 0-48 h}) of at least 3.9 ng/mL THC, at least 4.6 ng/mL 11-OH-THC, at least 50 ng/mL for 11-NOR-9-Carboxy-THC, or at least 1.2 ng/mL CBD; or
3) a mean time to maximum peak concentration (T_{max, 0-48 h}) of at most 1.5 h for THC, at most 2 h for 11-OH-THC, at most 2 h for 11-NOR-9-Carboxy-THC, or at most 1.5 h for CBD.

3. The cannabis oil emulsion of claim 1, wherein the administration of no less than 10 mg of CBD and no less than 10 mg of THC to a subject provides a bioavailability of CBD, 11-OH-THC and THC in plasma after the administration resulting in:
1) an average area under the curve (AUC_{0-48 h}) of at least a 11 h*ng/mL for THC, at least 26 h*ng/mL for 11-OH-THC, and at least 6 h*ng/mL for CBD;
2) an average maximum peak concentration (C_{max, 0-48 h}) of at least a 3.6 ng/mL THC, at least 4.2 ng/mL 11-OH-THC, and at least 1.4 ng/mL CBD; or
3) an average time to maximum peak concentration (T_{max, 0-48 h}) of at most 40 min for THC, at most 80 min for 11-OH-THC, and at most 40 min for CBD.

4. The cannabis oil emulsion of claim 1, wherein the administration of no less than 10 mg of CBD and no less than 10 mg of THC to a subject provides relative pharmacokinetic values for CBD, 11-OH-THC, 11-NOR-9-Carboxy-THC, and/or THC in plasma after the administration, when compared to the administration to a subject of a cannabis oil composition comprising CBD and THC dissolved in MCT oil (the Comparator formulation) comprising no less than 10 mg of CBD and no less than 10 mg of THC, resulting in:
1) a relative mean area under the curve (AUC_{0-48 h}) increase of the cannabis oil emulsion of claim 1 over the Comparator formulation of at least 25% for THC, at least 25% for 11-OH-THC, at least 25% for 11-NOR-9-Carboxy-THC, or at least 25% for CBD;
2) a relative mean maximum peak concentration (C_{max, 0-48 h}) increase of the cannabis oil emulsion of claim 1 over the Comparator formulation of at least 25% for THC, at least 25% for 11-OH-THC, at least 25% for 11-NOR-9-Carboxy-THC, or at least 25% for CBD; or
3) a relative mean time to maximum peak concentration (T_{max, 0-48 h}) reduction of the cannabis oil emulsion of claim 1 over the Comparator formulation of at least 25% for THC, at least 25% for 11-OH-THC, at least 25% for 11-NOR-9-Carboxy-THC, or at least 25% for CBD.

5. The cannabis oil emulsion of claim 1, wherein the emulsifier is GRAS or a food grade emulsifier.

6. The cannabis oil emulsion of claim 1, wherein the ratio of CBD:THC is 2:3 to 3:2.

7. The cannabis oil emulsion of claim 1, further comprising one or more co-solvents selected from the group consisting of ethanol, glycerol, propylene glycol, 1,3-propanediol, butylene glycol, erythritol, xylitol, mannitol, sorbitol, isomalt, polyethylene glycols, and a combination thereof.

8. The cannabis oil emulsion of claim 1, further comprising one or more vegetable oil selected from the group consisting of arachis oil, olive oil, sesame oil or coconut oil and a mineral oil, cannabis oil (hemp oil), coconut oil, cottonseed oil, soybean oil, sunflower oil, castor oil, corn oil, olive oil, palm oil, peanut oil, almond oil, sesame oil, rapeseed oil, peppermint oil, canola oil, palm kernel oil, hydrogenated soybean oil, medium-chain triglycerides (MCLs), short-chain triglycerides, glyceryl esters of saturated fatty acids, glyceryl behenate, glyceryl distearate, glyceryl isostearate, glyceryl aurate, glyceryl monooleate, glyceryl monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and combinations thereof.

9. The cannabis oil emulsion of claim 1, further comprising one or more masking or flavoring component selected from the group consisting of natural cinnamon oil, peppermint oil, clove oil, bay oil, thyme oil, artificial, natural or synthetic fruit flavors selected from the group consisting of vanilla, chocolate, coffee, cocoa, and citrus oil selected from the group consisting of lemon, lime, orange, grape, grapefruit, and fruit essences selected from the group consisting of apple, pear, peach, strawberry, watermelon, raspberry, cherry, plum, pineapple and apricot, or combinations thereof.

10. The cannabis oil emulsion of claim 1, further comprising one or more additives comprising:
a) a stabilizer or antioxidant selected from the group consisting of tocopherols, flavonoids, catechins, superoxide dismutase, lecithin, gamma oryzanol, vitamins A, C (ascorbic acid) and E including homologues and isomers thereof, camosol, carnosic acid and rosmanol, hawthorn extract and proanthocyanidins, or combinations thereof; and
b) a reducing agent selected from the group consisting of L-ascorbic acid-6-palmitate, vitamin C and ubiquinol, or mixtures thereof.

11. The cannabis oil emulsion of claim 1, further comprising a metal chelator selected from the group consisting of ethylenediaminetetraacetic acid (EDTA), disodium EDTA and calcium disodium EDTA and mixtures thereof.

12. The cannabis oil emulsion of claim 1, further comprising an absorption enhancer or bioavailability enhancer selected from the group consisting of medium chain fatty acids, omega-3 fatty acids, capric acid, caprylic acid, (8-[2-hydroxybenzoyl]-amino)caprylic acid, N-(10-[2-hydroxybenzoyl]-amino)decanoic acid, N-(8-[2-hydroxybenzoyl]-amino)caprylic acid (SNAC, salcaprozate sodium), 8-(N-2-hydroxy-5-chloro-benzoyl)-amino-caprylic acid (5-CNAC), N-(10-[2-hydroxybenzoyl]-amino)decanoic acid, alkylglycosides, chitosan, trimethylated chitosan, protease inhibitors, β-glycoprotein inhibitors, dodecyl-2-N,N-dimethylamino propionate (DDAIP), calcium chelating agents (i.e. ethylene glycol tetraacetic acid, ethylene diamine tetraacetic acid (EDTA), salicylic acid, flavonoids (i.e. quercetin ((2-(3,4-dihydroxyphenyl)-3,5,7-trihydroxy-4Hchromen-4-one), luteolin), isoflavones (i.e. genistein (5,7-Dihydroxy-3-(4-hydroxyphenyl)chromen-4-one)), flavonoid glycosides (naringin), alkaloids (i.e. sinomenine (7,8-didehydro-4-hydroxy-3,7-dimethoxy-17-methylmorphinan-6-one), triterpenoid saponins (glycyrrhizin [(3,18)-30-hydroxy-11,30-dioxoolean-12-en-3-yl 2-O-glucopyranuronosyl-Dglucopyranosiduronic acid]), nitrile glycosides, phytomolecules (i.e. lysergol, allicin (garlic)), terpenes (ginkgolide A, B, C and J), ginsenosides, epigallocatechin, epigallocatechin gallate, phenanthrene, cuminumcyminum Linn, herb, ginger, aloe vera, capsaicin, colchicine, vincristine, matrine, ammonium glycyrrhizinate, beeswax, piperine, trikatu, and their pharmaceutically acceptable salts (i.e. sodium), or derivatives (i.e. esters).

13. The cannabis oil emulsion of claim 1, wherein the range of the ratio of the emulsifier to CBD/THC is between 11.0:1.0 to 1.0:1.0, 7.0:1.0 to 1.5:1.0 or 5.0:1.0 to 2.0:1.0.

14. The cannabis oil emulsion of claim 1, wherein the CBD/THC concentration in the emulsion is about 10%, 9%, 8%, 7%, 5%, 3%, 2%, 1%, 0.5%, 0.1% or 0.01% or less.

15. The cannabis oil emulsion of claim 1, wherein the emulsion comprises of particle size is less than about 500 nm, less than 300 nm, less than 200 nm, less than 100 nm, less than 80 nm, less than 60 nm; less than 40 nm; or between about 20 and 30 nm, as measured by DLS.

16. The cannabis oil emulsion of claim 1, wherein the emulsion has a measured Nephelometric Turbidities in a range of about 10 to 1000, 20 to 300 or 30 to 100.

17. A method for the preparation of the cannabis oil emulsion of claim 1, the method comprising:
a) weighing the components of the emulsion of claim 1 into a reaction container;
b) heating the combined emulsion to a temperature from about 25° C. to about 130° C. with agitation for a sufficient amount of time to prepare the emulsion; and
c) cooling the emulsion to about 25° C.

18. The method of claim 17, wherein the heating of the cannabis oil emulsion is performed to a temperature of about 70° C. to 100° C., or about 80° C. to 95° C.

19. (canceled)

20. The cannabis oil emulsion prepared by the method of claim 17, wherein the resulting stable emulsion has a shelf stability of at least 3 months, 6 months or 12 months when stored at about 0° C. to 50° C., or about 25° C. to 35° C.

21. (canceled)

22. (canceled)

23. A liquid nutritional composition selected from the group consisting of beverages, soft drinks, carbonated beverages, enhanced waters, gels, gelatins, concentrates, beverage enhancers, wherein the composition is prepared by the method comprising:
a) obtaining the cannabis oil emulsion of claim 1; and
b) diluting the cannabis oil emulsion to a desired liquid nutritional composition.

24.-26. (canceled)
